(19) United States
(12) Patent Application Publication
(10) Pub. No.: US 2010/0255117 A1
(43) Pub. Date: Oct. 7, 2010

(54) METHODS AND COMPOSITIONS FOR THE TREATMENT OF CANCER

(75) Inventors: Shyam Biswal, Ellicott City, MD (US); Anju Singh, Baltimore, MD (US); Deepti Malhotra, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/594,936

(22) PCT Filed: Apr. 6, 2008
(86) PCT No.: PCT/US08/59520

§ 371 Date: Jun. 18, 2010

Related U.S. Application Data
(60) Provisional application No. 60/922,230, filed on Apr. 6, 2007.
(60) Provisional application No. 60/925,484, filed on Apr. 20, 2007.

(51) Int. Cl. A61K 33/42; A61P 35/00; A61K 31/375; C07D 307/62; C07C 279/26; A61K 31/155; C07D 491/08; A61K 31/46; C07C 235/46; A61K 31/609; A61K 33/30; C07D 495/04; A61K 31/4365; C07D 487/04; A61K 31/5517; C07D 295/096; A61K 31/495; A61K 31/65; C07C 237/26; C07J 3/00; A61K 31/5685; A61K 36/37; C08B 37/18; A61K 31/733; C07H 21/02; C12Q 1/66; C12Q 1/68; C01B 25/10; C01G 9/04

(52) U.S. Cl. 424/605; 514/474; 549/317; 564/235; 514/635; 546/91; 514/291; 564/177; 514/166; 424/641; 546/114; 514/301; 540/579; 514/214.02; 544/401; 514/255.03; 514/152; 552/200; 552/610; 514/180; 424/769; 536/123.1; 514/54; 514/44 A; 435/8; 435/6; 423/301; 423/491

(57) ABSTRACT

The instant invention provides methods and compositions for the treatment of cancer.

### RELATED APPLICATIONS

This application claims the benefit of U.S. Provisional Application No.: 60/922,230, filed Apr. 6, 2007 and U.S. Provisional Application No.: 60/925,484, filed Apr. 20, 2007. The entire contents of each of the aforementioned applications is hereby expressly incorporated herein.

### BACKGROUND

Cancers are one of the leading causes of death in humans. Despite the advances in cancer treatment, many cancers become resistant to standard chemotherapeutic or radiotherapeutic treatment regimes.

Lung cancer is the leading cause of cancer deaths in the United States and worldwide for men and women. Despite considerable progress over the last 25 years in the systemic therapy of lung cancer, intrinsic and acquired resistance to chemotherapeutic agents and radiation remains a challenge (Nadkar et al., 2006). Most patients with small cell lung cancer (SCLC) have an initial response to chemotherapy but the majority relapse and their tumors tend to be largely refractory to further treatment. Non-small-cell-lung cancers (NSCLC) are intrinsically resistant and are generally non-responsive to initial chemotherapy. Frequently, resistance is intrinsic to the cancer, but as the therapy becomes increasingly effective, acquired resistance has also become common (Nadkar et al., 2006).

Formation of reactive oxygen species (ROS) is important for induction of apoptosis for commonly used chemotherapy agents such as cisplatin, bleomycin, paclitaxel, adriamycin and etoposide (Kurosu et al., 2003; Masuda et al., 1994). Xenobiotic metabolism enzymes in conjunction with drug efflux proteins act to detoxify cancer drugs, whereas antioxidants confer cytoprotection by attenuating drug-induced oxidative stress and apoptosis. Several studies have shown that the expression of xenobiotic metabolism genes [glutathione-S-transferases (GSTs)], antioxidants [glutathione (GSH)], and drug efflux proteins [multidrug resistance protein (MRP) family] are increased in NSCLC (Soini et al., 2001; Tew, 1994; Yang et al., 2006). Ionizing radiation kills cancer cells by generation of reactive oxygen species (ROS), mainly superoxide, hydroxyl radicals and hydrogen peroxide which causes DNA damage, and upregulation of antioxidant enzyme expression or addition of free radical scavengers has been reported to protect cells from the damaging effects of radiation (Lee et al., 2004; Weiss and Landauer, 2003). Thus, radiations as well as widely used chemotherapeutic agents depend on oxidative insult to cancer cells for their mode of action. Cancer cells exhibit a superior defense system against electrophiles as compared with normal cells due to the upregulation of genes involved in electrophile detoxification. In addition, lung cancer cells have greater expression of multidrug resistance proteins which confer chemoresistance (Trachootham et al., 2006).

Intrinsic resistance to radio- and chemotherapy remains a challenge in most cancers. Cancer cells are endowed with aberrant transcriptional program for increased expression of antioxidants, drug detoxification and efflux genes that cause resistance to therapy.

Accordingly, a need exists for new and more effective cancer treatments.

### SUMMARY OF THE INVENTION

The instant invention is based, at least in part, on the discovery by the inventors that Nrf2 plays a major role in cancer progression and in the ability of cancer cells to become resistant to chemotherapeutic and radiation therapy.

Accordingly, in at least one aspect, the instant invention provides a Nrf2 inhibitor as set forth in Table 5. In one embodiment, the Nrf2 inhibitor as set forth in Table 5 is used for treating a cell proliferative disorder, e.g., cancer. In one embodiment, the cancer is a solid tumor cancer, e.g., lung, breast, or prostate cancer. In another embodiment, the cell proliferative disorder is a hematological cancer, e.g. leukemia, acute lymphoblastic leukemia (ALL), acute myelogenous leukemia (AML), chronic myelogenous leukemia (CML), chronic lymphocytic leukemia (CLL), hairy cell leukemia, or multiple myeloma.

In another aspect, the instant invention provides methods for identifying an inhibitor of Nrf2 comprising: contacting a carcinoma cell transfected with luciferase with a candidate inhibitor of Nrf2; and measuring the luciferase activity in the cells; wherein a decrease in the amount of luciferase activity as compared to a carcinoma cell transfected with luciferase not contacted with the candidate inhibitor is indicative of the candidate inhibitor being an inhibitor of Nrf2. In one embodiment, the carcinoma cell is a adenocarcinoma cell, e.g., a lung adenocarcinoma cell. In one embodiment, the carcinoma cell is contacted with the candidate inhibitor for at least 12 hours.

In one embodiment, the inhibitor is an antibody, peptide, polypeptide, nucleic acid, antisense molecule, siRNA, shRNA, microRNA, ribozyme, small molecule.

In another aspect, the invention provides a method of treating a subject having a cell proliferative disorder, comprising: administering to the subject an effective amount of a Nrf2 inhibitor; thereby treating the subject. In one embodiment, the subject is administered an additional anticancer treatment, e.g., radiation or a chemotherapeutic.

In one embodiment, the cancer is a solid tumor cancer, e.g., lung, breast, or prostate cancer. In another embodiment, the cell proliferative disorder is a hematological cancer, e.g. leukemia, acute lymphoblastic leukemia (ALL), acute myelogenous leukemia (AML), chronic myelogenous leukemia (CML), chronic lymphocytic leukemia (CLL), hairy cell leukemia, or multiple myeloma.

In one aspect, the instant invention provides methods for treating a subject having a cell proliferative disorder comprising: administering to the subject a Nrf2 inhibitor and one or more additional anticancer treatments, thereby treating the subject. In one embodiment, the anticancer treatment is radiation or a chemotherapeutic. In a related embodiment, the cell proliferative disorder is cancer. In one embodiment, the cancer is a solid tumor cancer, e.g., lung, breast, or prostate cancer. In another embodiment, the cell proliferative disorder is a hematological cancer, e.g. leukemia, acute lymphoblastic leukemia (ALL), acute myelogenous leukemia (AML), chronic myelogenous leukemia (CML), chronic lymphocytic leukemia (CLL), hairy cell leukemia, or multiple myeloma.

In one embodiment, the Nrf2 inhibitor is an antibody, peptide, polypeptide, nucleic acid, antisense molecule, siRNA, shRNA, microRNA, ribozyme, small molecule.

In another aspect, the invention provides methods for treating a subject having a cell proliferative disorder comprising: administering to the subject a compound that inhibits the expression or activity of Nrf2; thereby treating the subject.

In one embodiment, the cancer is a solid tumor cancer, e.g., lung, breast, or prostate cancer. In another embodiment, the cell proliferative disorder is a hematological cancer, e.g. leukemia, acute lymphoblastic leukemia (ALL), acute myelogenous leukemia (AML), chronic myelogenous leukemia (CML), chronic lymphocytic leukemia (CLL), hairy cell leukemia, or multiple myeloma. In one embodiment, the Nrf2 inhibitor is an antibody, peptide, polypeptide, nucleic acid, antisense molecule, siRNA, shRNA, microRNA, ribozyme, small molecule.

In a related embodiment, the compound that inhibits the activity or expression of Nrf2 is administered with a second anticancer treatment, e.g., radiation or a chemotherapeutic.

In another aspect, the instant invention provides method for determining if a subject is at risk of becoming resistant to an anticancer treatment comprising: determining if a subject has a mutation in the KEAP1 gene; thereby determining if a subject is at risk of developing resistance to anticancer treatment. In a related embodiment, the anticancer treatment is a chemotherapeutic or radiation.

In a specific embodiment, the mutation results in an amino acid substitution, e.g., at position 255 of KEAP1 (SEQ ID NO:3). In one embodiment, the substitution at position 255 is a Tyr to His mutation.

In a specific embodiment, the mutation results in an amino acid substitution, e.g., at position 314 of KEAP1 (SEQ ID NO:3). In one embodiment, the substitution at position 314 is a Thr to Met mutation. In a related embodiment, the subject's treatment is managed based on presence of a KEAP1 mutation.

In another aspect, the invention provides pharmaceutical compositions for the treatment of cancer comprising a Nrf2 inhibitor and a pharmaceutically acceptable carrier. In one embodiment, the Nrf2 inhibitor is set forth in Table 5. In an further embodiment, the pharmaceutical composition further comprises one or more additional anticancer compositions.

In a related embodiment, the invention provides pharmaceutical compositions comprising one or more Nrf2 inhibitors, one or more additional anticancer compositions and a pharmaceutically acceptable carrier.

In another aspect the instant invention provides kits for identifying inhibitors of Nrf2 comprising a carcinoma cell transfected with luciferase and instructions for use. In a related embodiment, the kits further comprise reagents for a luciferase assay.

### BRIEF DESCRIPTION OF THE DRAWINGS

FIGS. 1A-C depict the generation of cell lines stably expressing NRF2 shRNA. (A-B) Real time RT-PCR analysis of NRF2 expression in A549 and H460 cells stably expressing NRF2 shRNA. Total RNA from stable clones harboring NRF2 shRNA or non-targeting luciferase shRNA were analyzed for expressing of NRF2. GAPDH was used as normalization control. (C). Immunoblot detection of NRF2 in A549 and H460 cells stably transfected with shRNAs targeting NRF2. Cellular lysates of A549 (100 μg) and H460 (75 μg) were separated by SDS-PAGE and NRF2 was detected by immunoblotting with anti-NRF2 antibody.

FIGS. 2A-C depict inhibition of NRF2 activity leads to ROS accumulation in A549-NRF2shRNA and H460-NRf2shRNA cells. (A-B) Comparison of ROS levels in A549 and H460 cells stably expressing NRF2 shRNA. Cells expressing non-targeting Luc shRNA were used as control. Pretreatment with 20 mM NAC decreased the ROS levels. ROS levels in cells expressing luciferase shRNA were same as the control untransfected cells. (C) ROS levels did not change significantly between the BEAS2B cells transfected with NRF2 siRNA and the control non-targeting NS siRNA. *, p<0.01 relative to the cells expressing luciferase shRNA; **, p<0.01 relative to the cells pretreated with NAC.

FIGS. 3A-D Overexpression of NRF2 confers drug resistance. (A-D) Enhanced sensitivity of NRF2 shRNA expressing A549 and H460 cells to carboplatin and etoposide. Cells were exposed to drugs for 72 h-96 h and viable cells were determined by MTS/phenazine methosulfate assay. Data is represented as percentage of viable cells relative to the vehicle treated control. Data are mean of 8 independent replicates, combined to generate the mean±SD for each concentration. Representative experiments are shown.

FIGS. 4A-D depict inhibition of NRF2 activity confers sensitivity to ionizing radiation. (A-B) Clonogenic survival of A549 and H460 cells stably expressing NRF2 shRNA. Cells expressing non-targeting Luc shRNA were used as control. (C&D) Pretreatment with NAC decreased the radiation induced cytotoxicity in A549 and h460 cells stably expressing NRF2 shRNA. *, p<0.01 relative to the cells expressing luciferase shRNA at the same radiation dose; **, p<0.01 relative to the cells exposed to gamma radiation without pretreatment with NAC.

FIGS. 5A-G depict NRF2 ablation leads to reduced tumorigenic properties in vitro and in vivo. (A-B) NRF2 promotes lung cancer cell proliferation. A549-NRF2shRNA (1500 cells) and H460-NRF2shRNA (1000 cells) cells were plated in 96 well plates and cellular proliferation was analyzed using the colorimetric MTS assay over the indicated time course. Cancer cells expressing Luc-shRNA were used as control. (C) A549-NRF2shRNA and H460-NRF2shRNA expressing cells were also analyzed for anchorage-independent growth. (D-G) A549-NRF2shRNA and H460-NRF2shRNA cells were injected in the flank of male athymic nude mice (n=7 for H460, n=6 for A549). A549 and H460 cells expressing Luc-shRNA were used as control. Weekly measurements were taken from the tumors, and the mean tumor volume was determined after 4-6 weeks. Weight of the tumor was recorded at the termination of the experiment. Mean difference in tumor weight between the Luc-shRNA and NRF2 shRNA expressing H460 cells was 1.24 gms (95% CI=0.773 to 1.71; P=0.0001). Data was analyzed using two-sample Wilcoxon rank-sum (Mann-Whitney) test. A549-NRF2 shRNA cells did not form any tumor in nude mice.

FIGS. 6A-B depict therapeutic efficacy of NRF2 siRNA in combination with carboplatin and radiation. (A) Nude mice were injected subcutaneously with A549 cells and randomly allocated to one of the following groups with therapy beginning 15 days after tumor cell injection: GFP siRNA, GFP siRNA+carboplatin, GFP siRNA+radiation, NRF2 siRNA, NRF2 siRNA+carboplatin and NRF2 siRNA+radiation. Mice were treated for 4 weeks and then sacrificed. A dot plot shows the tumor weights upon termination by treatment group. Weights of the GFP siRNA treated tumors were significantly higher compared to NRF2 siRNA treated tumors (p=0.01), and siRNA treated compared to siRNA+carboplatin treated tumors (p=0.001). There were no significant differences in tumor weights between siRNA+radiation and siRNA+carboplatin treated tumors (p=0.40). (B) Delivery of naked NRF2 siRNA duplex into tumor inhibited the expression of NRF2 and its downstream target genes (HO-1 and GCLm). ‘*’,P<0.05 (Wilcoxon rank-sum test).

FIGS. 7A-H depict delivery of naked siRNA duplexes into orthotopic lung tumors. (A-B) Mice were injected with Lewis lung carcinoma cells and 24 days later (when the mice developed larger tumors) mice were inhaled for three consecutive days with 100 μg/day/mouse of Cy3 labeled naked chemically stabilized reference siRNA using a nebulizer. Twenty four hours after last siRNA administration, mice were sacrificed; lungs harvested and sectioned. Resulting sections were analyzed by Bio-Rad Confocal microscope using a 20×Water objective and 2×zoom combined to give a total of 40×magnification. Control, non-siRNA-treated lungs were used to set up background fluorescence level. Green—background fluorescence, red—Cy3-siRNA. (A)

Localization of Cy3 labeled siRNA in a large surface tumor. (B) Localization of labeled siRNA in intraparenchymal tumor. The large surface-protruding tumors showed Cy3 signal but the intensity was several folds lower than that observed in the small intra-parenchymal tumors. (C-F) Delivery of NRF2 siRNA into A549 lung tumors. A549 cells stably expressing luciferase reporter were injected into SCID-Beige mice via tail vein. Mice were randomly allocated to one of the following groups (n=5/group) with siRNA inhalations and carboplatin treatment beginning 1 week after tumor cell injection: GFP siRNA, GFP siRNA+carboplatin, NRF2 siRNA and NRF2 siRNA+carboplatin. After 4 weeks of treatment, mice were imaged using Xenogen imaging system and luciferin substrate. (G) A dot plot shows the distribution of lung weights upon termination by treatment group. The weights did not vary significantly between overall treatment groups of

GFP siRNA and NRF2 siRNA. However, the lung weights for siRNA treated tumors were significantly higher than for siRNA+carboplatin treated tumors (ratio of weights=1.73 [1.46, 2.06], p=0.0001). The difference in weights between siRNA and siRNA+carboplatin treated tumors was significant between NRF2 siRNA and GFP siRNA treated tumors (1.46, 95% CI: [1.03, 2.09], p=0.05). (H) A scatter plot of ventral view flux (evaluated by in vivo Xenogen imaging) and lung weights upon termination.

FIG. 8 depicts Table 1 showing the list of genes downregulated in A549-NRF2shRNA and H460-NRF2 shRNA cells in response to NRF2 inhibition. The expressions of several NRF2 dependent genes were quantified using real time RT-PCR. Cells stably expressing luciferase shRNA were used as baseline control to calculate the fold changes. All the represented fold change values of NRF2 siRNA transfected cells or NRF2 shRNA expressing cells are significant compared to the control cells transfected with luciferase.

FIG. 9 depicts Table 2 showing mean (SD) of subcutaneous tumor weights and changes in tumor volume by treatment group for experiment-1.

FIG. 10 depicts Table 3 showing mean (SD) of subcutaneous tumor weights and changes in tumor volume by treatment group for experiment-2.

FIG. 11 is Table 4 which depicts mean (SD) lung weights by treatment groups for lungs from SCID beige mice injected with A549 cells.

FIGS. 12A-F depict the comparison of GSR, GPX, GST, G6PDH and total GSH levels between cells expressing NRF2 shRNA and control cells expressing luciferase shRNA. Shown are enzyme activities for GSR (A), GPX (B), GST (C) total GSH levels (D) and G6PDH (E). Data represent mean±SE (n=3). *, p<0.05 relative to the cells expressing luciferase shRNA (by t-test). (F) Western blot analysis of TXN and TXNRD1 levels in A549 cells stable transfected with the NRF2 shRNA and control cells expressing luciferase shRNA.

FIGS. 13A-D depicts the effect of NRF2 shRNA on drug accumulation in lung cancer cells. (A-D) Tritium (³H) labeled etoposide and ¹⁴C labeled carboplatin accumulation in A549-NRF2shRNA and H460-NRF2shRNA cells was measured after 60 min and 120 mins of incubation with the drug. A non-targeting luciferase shRNA with microarray defined signature was used as control. Data are mean of 3 independent replicates, combined to generate the mean±SE for each concentration. Drug accumulation was significantly higher in cells expressing NRF2 shRNA. *, P<0.01 relative to Luc shRNA.

FIG. 14 depicts a dot plot showing the tumor weights by treatment group from second experiment. Tumor weights were significantly higher in the GFP tumors compared to the NRF2 tumors (ratio of tumor weights=2.80, 95% CI: [1.71, 4.60], p=0.0009) and lower in the siRNA+carboplatin treated tumors compared to the siRNA treated tumors (0.55, 95% CI: [0.33, 0.91], p=0.033). The difference in tumor weights between treatment groups was not significantly different between NRF2 and GFP tumors (interaction p=0.70).

FIG. 15 depicts SCID-Beige mice injected i.v. with ARE-luciferase reporter tumor cells were inhaled NRF2 siRNA-2 twice during the 4th week of lung tumor growth. Control mice were inhaled GFP siRNA. Mice were imaged before and after siRNA inhalation.

FIG. 16 depicts Table 5 demonstrating Nrf2 inhibitors identified in the assay described in Example 2. The middle column identifies the known use of each compound, and the right hand column depicts the percent inhibition of luciferase activity for each compound.

FIG. 17 depicts KEAP1 miRNA hsa-miR-125b.

FIGS. 18A-B depict the amino acid and nucleic acid sequence of human Nrf2 (SEQ ID NO:1 and 2, respectively).

FIGS. 19A-B depict the amino acid and nucleic acid sequence of human KEAP1. The sequences of two variants of KEAP1 are provided. Accordingly, KEAP1 amino acid sequences for variants 1 and 2 are set forth as SEQ ID NO: 3 and 5, respectively. KEAP1 nucleic acid sequences for variants 1 and 2 are set forth as SEQ ID NO: 4 and 6, respectively.

### DETAILED DESCRIPTION OF THE INVENTION

The instant invention is based, at least in part, on the discovery that Nrf2 is a global regulator of cancer. Moreover, the instant inventors have discovered that increased NRF2 function in cancer cells promotes tumorigenicity and contributes to subjects becoming resistant to chemotherapeutics and radiation treatment. The inventors also provide methods for identifying compounds that inhibit Nrf2 and methods of treating subjects having cell proliferative disorders. Moreover, the inventors have discovered that mutations in KEAP1, a constitutive suppressor of Nrf2 activity, are indicative of subjects becoming resistant to chemotherapeutic or radiation treatment

The instant invention is directed to methods and compositions for treating cell proliferative disorders, e.g., cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human cancer.

The instant invention provides methods for screening of Nrf2 inhibitors.

### Screening Assays

The invention provides a method (also referred to herein as a “screening assay”) for identifying modulators, i.e., candidate or test compounds or agents (e.g., peptides, peptidomimetics, nucleic acids, siRNAs, shRNAs, microRNAs, small molecules, or other drugs) that bind to Nrf2 proteins or have an inhibitory effect on, for example, Nrf2 expression or Nrf2 activity.

The test compounds, also referred to herein as “candidate inhibitor” of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind and or inhibit Nrf2 protein can be accomplished by a variety of methods. In one embodiment, the test compounds can be assayed for the ability to inhibit Nrf to using luciferase transfected cancer cells as described in the examples. Additionally, the assay could be conducted by coupling the test compound with a radioisotope or enzymatic label such that binding of the test compound to the Nrf2 protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In one embodiment, the assay components described herein can be packaged into a kit along with instructions for use. For example, the luciferase transfected cancer cells can be included in a kit comprising instructions for determining if a candidate compound is a Nrf2 inhibitor.

In a similar manner, one may determine the ability of the Nrf2 protein to bind to or interact with a Nrf2 target molecule. By “target molecule” is intended a molecule with which a Nrf2 protein binds or interacts in nature, e.g., KEAP1. In a preferred embodiment, the ability of the Nrf2 protein to bind to or interact with a Nrf2 target molecule can be determined by monitoring the activity of the target molecule. Also for example, the activity of the target molecule can be monitored by detecting induction of a cellular second messenger of the target, detecting catalytic/enzymatic activity of the target on an appropriate substrate, detecting the induction of a reporter gene (e.g., a kinase-responsive regulatory element operably linked to a nucleic acid encoding a detectable marker, e.g., luciferase), or detecting a cellular response, for example, cellular differentiation or cell proliferation.

In yet another embodiment, an assay of the present invention is a cell-free assay comprising contacting a Nrf2 protein or biologically active portion thereof with a test compound and determining the ability of the test compound to bind to the Nrf2 protein or biologically active portion thereof. Binding of the test compound to the Nrf2 protein can be determined either directly or indirectly as described above. In a preferred embodiment, the assay includes contacting the Nrf2 protein or biologically active portion thereof with a known compound that binds Nrf2 protein to form an assay mixture, contacting the assay mixture with a test compound, and determining the ability of the test compound to preferentially bind to Nrf2 protein or biologically active portion thereof as compared to the known compound.

In another embodiment, an assay is a cell-free assay comprising contacting Nrf2 protein or biologically active portion thereof with a test compound and determining the ability of the test compound to modulate (e.g., stimulate or inhibit) the activity of the Nrf2 protein or biologically active portion thereof. Determining the ability of the test compound to modulate the activity of a Nrf2 protein can be accomplished, for example, by determining the ability of the Nrf2 protein to bind to a Nrf2 target molecule as described above for determining direct binding. In an alternative embodiment, determining the ability of the test compound to modulate the activity of a Nrf2 protein can be accomplished by determining the ability of the Nrf2 protein to further modulate a Nrf2 target molecule. For example, the catalytic/enzymatic activity of the target molecule on an appropriate substrate can be determined as previously described.

In yet another embodiment, the cell-free assay comprises contacting the Nrf2 protein or biologically active portion thereof with a known compound that binds a Nrf2 protein to form an assay mixture, contacting the assay mixture with a test compound, and determining the ability of the test compound to preferentially bind to or modulate the activity of a Nrf2 target molecule.

In the above-mentioned assays, it may be desirable to immobilize either a Nrf2 protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/Nrf2 fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or Nrf2 protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of Nrf2 binding or activity determined using standard techniques.

Other techniques for immobilizing proteins on matrices can also be used in the screening assays of the invention. For example, either Nrf2 protein or its target molecule can be immobilized utilizing conjugation of biotin and streptavidin. Biotinylated Nrf2 molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a Nrf2 protein or target molecules but which do not interfere with binding of the Nrf2 protein to its target molecule can be derivatized to the wells of the plate, and unbound target or Nrf2 protein trapped in the wells by antibody conjugation. Methods for detecting such complexes, in addition to those described above for the GST-immobilized complexes, include immunodetection of complexes using antibodies reactive with the Nrf2 protein or target molecule, as well as enzyme-linked assays that rely on detecting an enzymatic activity associated with the Nrf2 protein or target molecule.

In another embodiment, modulators of Nrf2 expression are identified in a method in which a cell is contacted with a candidate compound and the expression of Nrf2 mRNA or protein in the cell is determined relative to expression of Nrf2 mRNA or protein in a cell in the absence of the candidate compound. When expression is greater (statistically significantly greater) in the presence of the candidate compound than in its absence, the candidate compound is identified as a stimulator of Nrf2 mRNA or protein expression. Alternatively, when expression is less (statistically significantly less) in the presence of the candidate compound than in its absence, the candidate compound is identified as an inhibitor of Nrf2 mRNA or protein expression. The level of Nrf2 mRNA or protein expression in the cells can be determined by methods described herein for detecting Nrf2 mRNA or protein.

In yet another aspect of the invention, the Nrf2 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with Nrf2 protein (“Nrf2-binding proteins” or “Nrf2-bp”) and modulate Nrf2 activity. Such Nrf2-binding proteins are also likely to be involved in the propagation of signals by the Nrf2 proteins as, for example, upstream or downstream elements of the Nrf2 pathway.

This invention further pertains to novel agents identified by the above-described screening assays and uses thereof for treatments as described herein.

### Molecules of the Invention

Nrf2 proteins are also encompassed within the present invention. By “Nrf2 protein” is intended a protein having the amino acid sequence set forth in SEQ ID NO: 2, as well as fragments, biologically active portions, and variants thereof.

KEAP1 proteins are also useful in the methods of the invention. By “KEAP1 protein” is intended a protein having the amino acid sequence set forth in SEQ ID NO: 4, as well as fragments, biologically active portions, and variants thereof.

“Fragments” or “biologically active portions” include polypeptide fragments suitable for use as immunogens to raise antibodies. Fragments include peptides comprising amino acid sequences sufficiently identical to or derived from the amino acid sequence of a protein, or partial-length protein, of the invention and exhibiting at least one activity of the protein, but which include fewer amino acids than the full-length, e.g., less than the full-length of SEQ ID NO:2. Typically, biologically active portions comprise a domain or motif with at least one activity of the protein. A biologically active portion of Nrf2 can be a polypeptide which is, for example, 10, 25, 50, 100 or more amino acids in length.

Antibodies

The invention also provides Nrf2 antibodies. An isolated Nrf2 polypeptide of the invention can be used as an immunogen to generate antibodies that bind Nrf2 proteins using standard techniques for polyclonal and monoclonal antibody preparation. The full-length Nrf2 protein can be used or, alternatively, the invention provides antigenic peptide fragments of Nrf2 proteins for use as immunogens. The antigenic peptide of a Nrf2 protein comprises at least 8, preferably 10, 15, 20, or 30 amino acid residues of the amino acid sequence shown in SEQ ID NO:2 and encompasses an epitope of a Nrf2 protein such that an antibody raised against the peptide forms a specific immune complex with the Nrf2 protein. Preferred epitopes encompassed by the antigenic peptide are regions of a Nrf2 protein that are located on the surface of the protein, e.g., hydrophilic regions.

Accordingly, another aspect of the invention pertains to anti-Nrf2 polyclonal and monoclonal antibodies that bind a Nrf2 protein. Polyclonal anti-Nrf2 antibodies can be prepared by immunizing a suitable subject (e.g., rabbit, goat, mouse, or other mammal) with a Nrf2 immunogen. The anti-Nrf2 antibody titer in the immunized subject can be monitored over time by standard techniques, such as with an enzyme linked immunosorbent assay (ELISA) using immobilized Nrf2 protein. At an appropriate time after immunization, e.g., when the anti-Nrf2 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-Nrf2 antibody can be identified and isolated by screening a recombinant combinatorial immunoglobulin library (e.g., an antibody phage display library) with a Nrf2 protein to thereby isolate immunoglobulin library members that bind the Nrf2 protein. Kits for generating and screening phage display libraries are commercially available (e.g., the Pharmacia Recombinant Phage Antibody System, Catalog No. 27-9400-01; and the Stratagene SurfZap□ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-Nrf2 antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985)

Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994)

Bio/Technology 12:899-903).

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

Antisense Molecules

In one embodiment, the Nrf2 inhibitor is an antisense molecule. Antisense molecules as used herein include antisense or sense oligonucleotides comprising a single-stranded nucleic acid sequence (either RNA or DNA) capable of binding to target mRNA (sense) or DNA (antisense) sequences for cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules can be modified or unmodified RNA, DNA, or mixed polymer oligonucleotides. These molecules function by specifically binding to matching sequences resulting in inhibition of peptide synthesis (Wu-Pong, November 1994, BioPharm, 20-33) either by steric blocking or by activating an RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190). In addition, binding of single stranded DNA to RNA can result in nuclease-mediated degradation of the heteroduplex (Wu-Pong, supra). Backbone modified DNA chemistry which have thus far been shown to act as substrates for RNase H are phosphorothioates, phosphorodithioates, borontrifluoridates, and 2′-arabin and 2′-fluoro arabino-containing oligonucleotides.

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

RNAi

RNA interference refers to the process of sequence-specific post transcriptional gene silencing in animals mediated by short interfering RNAs (siRNA) (Fire et al., Nature, 391, 806 (1998)). The corresponding process in plants is referred to as post transcriptional gene silencing or RNA silencing and is also referred to as quelling in fungi. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L. (reviewed in Sharp, P. A., RNA interference-2001, Genes & Development 15:485-490 (2001)).

Small interfering RNAs (siRNAs) are powerful sequence-specific reagents designed to suppress the expression of genes in cultured mammalian cells through a process known as RNA interference (RNAi). Elbashir, S. M. et al. Nature 411:494-498 (2001); Caplen, N. J. et al. Proc. Natl. Acad. Sci. USA 98:9742-9747 (2001); Harborth, J. et al. J. Cell Sci. 114:4557-4565 (2001). The term “short interfering RNA” or “siRNA” refers to a double stranded nucleic acid molecule capable of RNA interference “RNAi”, (see Kreutzer et al., WO 00/44895; Zernicka-Goetz et al. WO 01/36646; Fire, WO 99/32619; Mello and Fire, WO 01/29058). As used herein, siRNA molecules are limited to RNA molecules but further encompasses chemically modified nucleotides and non-nucleotides. siRNA gene-targeting experiments have been carried out by transient siRNA transfer into cells (achieved by such classic methods as liposome-mediated transfection, electroporation, or microinjection).

Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi. When introduced into a cell, they assemble with yet-to-be-identified proteins of an endonuclease complex (RNA-induced silencing complex), which then guides target mRNA cleavage. As a consequence of degradation of the targeted mRNA, cells with a specific phenotype characteristic of suppression of the corresponding protein product are obtained. The small size of siRNAs, compared with traditional antisense molecules, prevents activation of the dsRNA-inducible interferon system present in mammalian cells. This avoids the nonspecific phenotypes normally produced by dsRNA larger than 30 base pairs in somatic cells.

Intracellular transcription of small RNA molecules is achieved by cloning the siRNA templates into RNA polymerase III (Pol III) transcription units, which normally encode the small nuclear RNA (snRNA) U6 or the human RNase P RNA H1. Two approaches have been developed for expressing siRNAs: in the first, sense and antisense strands constituting the siRNA duplex are transcribed by individual promoters (Lee, N. S. et al. Nat. Biotechnol. 20, 500-505 (2002). Miyagishi, M. & Taira, K. Nat. Biotechnol. 20, 497-500 (2002).); in the second, siRNAs are expressed as fold-back stem-loop structures that give rise to siRNAs after intracellular processing (Paul, C. P. et al. Nat. Biotechnol. 20:505-508 (2002)). The endogenous expression of siRNAs from introduced DNA templates is thought to overcome some limitations of exogenous siRNA delivery, in particular the transient loss of phenotype. U6 and H1 RNA promoters are members of the type III class of Pol III promoters. (Paule, M. R. & White, R. J. Nucleic Acids Res. 28, 1283-1298 (2000)).

Co-expression of sense and antisense siRNAs mediate silencing of target genes, whereas expression of sense or antisense siRNA alone do not greatly affect target gene expression. Transfection of plasmid DNA, rather than synthetic siRNAs, may appear advantageous, considering the danger of RNase contamination and the costs of chemically synthesized siRNAs or siRNA transcription kits. Stable expression of siRNAs allows new gene therapy applications, such as treatment of persistent viral infections. Considering the high specificity of siRNAs, the approach also allows the targeting of disease-derived transcripts with point mutations, such as RAS or TP53 oncogene transcripts, without alteration of the remaining wild-type allele. Finally, by high-throughput sequence analysis of the various genomes, the DNA-based methodology may also be a cost-effective alternative for automated genome-wide loss-of-function phenotypic analysis, especially when combined with miniaturized array-based phenotypic screens. (Ziauddin, J. & Sabatini, D. M. Nature 411:107-110 (2001)).

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409:363 (2001)). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., Science, 293, 834 (2001)). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., Genes Dev., 15, 188 (2001)).

This invention provides an expression system comprising an isolated nucleic acid molecule comprising a sequence capable of specifically hybridizing to the CA sequences. In an embodiment, the nucleic acid molecule is capable of inhibiting the expression of the CA protein. A method of inhibiting expression of CA inside a cell by a vector-directed expression of a short RNA which short RNA can fold in itself and create a double strand RNA having CA mRNA sequence identity and able to trigger posttranscriptional gene silencing, or RNA interference (RNAi), of the CA gene inside the cell. In another method a short double strand RNA having CA mRNA sequence identity is delivered inside the cell to trigger posttranscriptional gene silencing, or RNAi, of the CA gene. In various embodiments, the nucleic acid molecule is at least a 7 mer, at least a 10 mer, or at least a 20 mer. In a further embodiment, the sequence is unique.

MicroRNA

The term “miRNA” is used according to its ordinary and plain meaning and refers to a microRNA molecule found in eukaryotes that is involved in RNA-based gene regulation. The term will be used to refer to the single-stranded RNA molecule processed from a precursor. Individual miRNAs have been identified and sequenced in different organisms, and they have been given names. Names of miRNAs and their sequences are provided herein. Additionally, other miRNAs are known to those of skill in the art and can be readily implemented in embodiments of the invention. The methods and compositions should not be limited to miRNAs identified in the application, as they are provided as examples, not necessarily as limitations of the invention.

MicroRNA molecules (“miRNAs”) are generally 21 to 22 nucleotides in length, though lengths of 17 and up to 25 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved by an enzyme called Dicer in animals. Dicer is ribonuclease III-like nuclease. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation. SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target. The present invention concerns, in some embodiments of the invention, short nucleic acid molecules that function as miRNAs or as inhibitors of miRNA in a cell. The term “short” refers to a length of a single polynucleotide that is 150 nucleotides or fewer. The nucleic acid molecules are synthetic. The term “synthetic” means the nucleic acid molecule is isolated and not identical in sequence (the entire sequence) and/or chemical structure to a naturally-occurring nucleic acid molecule, such as an endogenous precursor miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occuring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together.

It is understood that a “synthetic nucleic acid” of the invention means that the nucleic acid does not have a chemical structure or sequence of a naturally occuring nucleic acid. Consequently, it will be understood that the term “synthetic miRNA” refers to a “synthetic nucleic acid” that functions in a cell or under physiological conditions as a naturally occuring miRNA.

While many of the embodiments of the invention involve synthetic miRNAs or synthetic nucleic acids, in some embodiments of the invention, the nucleic acid molecule(s) need not be “synthetic.” In certain embodiments, a non-synthetic miRNA employed in methods and compositions of the invention may have the entire sequence and structure of a naturally occurring miRNA precursor or the mature miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not an miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

In other embodiments of the invention, there are synthetic nucleic acids that are miRNA inhibitors. An miRNA inhibitor is between about 17 to 25 nucleotides in length and comprises a 5′ to 3′ sequence that is at least 90% complementary to the 5′ to 3′ sequence of a mature miRNA. In certain embodiments, an miRNA inhibitor molecule is 17, 18, 19, 20, 21, 22, 23, 24, or 25 nucleotides in length, or any range derivable therein. Moreover, an miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. Probe sequences for miRNAs are disclosed in the appendix. While they have more sequence than an miRNA inhibitor, one of skill in the art could use that portion of the probe sequence that is complementary to the sequence of a mature miRNA as the sequence for an miRNA inhibitor. Table 1 indicates what the mature sequence of an miRNA is. Moreover, that portion of the probe sequence can be altered so that it is still 90% complementary to the sequence of a mature miRNA.

In some embodiments, of the invention, a synthetic miRNA contains one or more design elements. These design elements include, but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

miRNAs are apparently active in the cell when the mature, single-stranded RNA is bound by a protein complex that regulates the translation of mRNAs that hybridize to the miRNA. Introducing exogenous RNA molecules that affect cells in the same way as endogenously expressed miRNAs requires that a single-stranded RNA molecule of the same sequence as the endogenous mature miRNA be taken up by the protein complex that facilitates translational control. A variety of RNA molecule designs have been evaluated. Three general designs that maximize uptake of the desired single-stranded miRNA by the miRNA pathway have been identified. An RNA molecule with an miRNA sequence having at least one of the three designs is referred to as a synthetic miRNA.

Synthetic miRNAs of the invention comprise, in some embodiments, two RNA molecules wherein one RNA is identical to a naturally occurring, mature miRNA. The RNA molecule that is identical to a mature miRNA is referred to as the active strand. The second RNA molecule, referred to as the complementary strand, is at least partially complementary to the active strand. The active and complementary strands are hybridized to create a double-stranded RNA, called the synthetic miRNA, that is similar to the naturally occurring miRNA precursor that is bound by the protein complex immediately prior to miRNA activation in the cell. Maximizing activity of the synthetic miRNA requires maximizing uptake of the active strand and minimizing uptake of the complementary strand by the miRNA protein complex that regulates gene expression at the level of translation. The molecular designs that provide optimal miRNA activity involve modifications to the complementary strand.

Two designs incorporate chemical modifications in the complementary strand. The first modification involves creating a complementary RNA with a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules including NH2, NHCOCH₃, biotin, and others.

The second chemical modification strategy that significantly reduces uptake of the complementary strand by the miRNA pathway is incorporating nucleotides with sugar modifications in the first 2-6 nucleotides of the complementary strand. It should be noted that the sugar modifications consistent with the second design strategy can be coupled with 5′ terminal modifications consistent with the first design strategy to further enhance synthetic miRNA activities.

The third synthetic miRNA design involves incorporating nucleotides in the 3′ end of the complementary strand that are not complementary to the active strand. Hybrids of the resulting active and complementary RNAs are very stable at the 3′ end of the active strand but relatively unstable at the 5′ end of the active strand. Studies with siRNAs indicate that 5′ hybrid stability is a key indicator of RNA uptake by the protein complex that supports RNA interference, which is at least related to the miRNA pathwy in cells. The inventors have found that the judicious use of mismatches in the complementary RNA strand significantly enhances the activity of the synthetic miRNA.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with an miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

The invention also provides miRNAs targeting KEAP1. In specific embodiments, hsa-miR-125b, hsa-miR-491 and has-miR-141 are provided. These miRNA's inhibit KEAP1 activity leading to activation of NRF2 pathway.

Antimers or small molecules targeting KEAP1 miRNA also can be used to inhibit NRF2 activity.

### Pharmaceutical Compositions and Methods

In one embodiment, a method of inhibiting cancer cell division is provided. In another embodiment, a method of inhibiting tumor growth is provided. In a further embodiment, methods of treating cells or individuals with cancer are provided.

The method comprises administration of a cancer inhibitor. In particular embodiments, the cancer inhibitor is a nucleic acid molecule, a pharmaceutical composition, a therapeutic agent or small molecule, or a monoclonal, polyclonal, chimeric or humanized antibody. In further embodiments, the cancer inhibitors are administered in a pharmaceutical composition.

Pharmaceutical compositions encompassed by the present invention include as active agent, the polypeptides, polynucleotides, siRNA, shRNA, miRNA, antisense oligonucleotides, or antibodies of the invention disclosed herein in a therapeutically effective amount. An “effective amount” is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

The compositions can be used to treat cancer. In addition, the pharmaceutical compositions can be used in conjunction with conventional methods of cancer treatment, e.g., to sensitize tumors to radiation or conventional chemotherapy. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Where the pharmaceutical composition comprises an antibody that specifically binds to a gene product encoded by a differentially expressed polynucleotide, the antibody can be coupled to a drug for delivery to a treatment site or coupled to a detectable label to facilitate imaging of a site comprising cancer cells, such as prostate cancer cells. Methods for coupling antibodies to drugs and detectable labels are well known in the art, as are methods for imaging using detectable labels.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. Once formulated, the compositions contemplated by the invention can be (1) administered directly to the subject (e.g., as polynucleotide, polypeptides, small molecule agonists or antagonists, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 .mu.g to about 2 mg, about 5 .mu.g to about 500 .mu.g, and about 20 .mu.g to about 100 .mu.g of DNA can also be used during a gene therapy protocol. Factors such as method of action (e.g., for enhancing or inhibiting levels of the encoded gene product) and efficacy of transformation and expression are considerations that will affect the dosage required for ultimate efficacy of the antisense subgenomic polynucleotides. Where greater expression is desired over a larger area of tissue, larger amounts of antisense subgenomic polynucleotides or the same amounts re-administered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5, 219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24):11581.Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The administration of the inhibitors of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly.

In a preferred embodiment, the inhibitors are administered as therapeutic agents, and can be formulated as outlined above. Similarly, genes (including both the full-length sequence, partial sequences, or regulatory sequences of the coding regions) can be administered in gene therapy applications, as is known in the art. These genes can include antisense applications, either as gene therapy (i.e. for incorporation into the genome) or as antisense compositions, as will be appreciated by those in the art.

Thus, in one embodiment, methods of modulating Nrf2 gene activity in cells or organisms are provided. In one embodiment, the methods comprise administering to a cell an anti-Nrf2 antibody that reduces or eliminates the biological activity of an endogenous Nrf2 protein. Alternatively, the methods comprise administering to a cell or organism a recombinant nucleic acid encoding a Nrf2 protein. As will be appreciated by those in the art, this may be accomplished in any number of ways.

The instant invention provides methods and compositions for inhibiting the development of resistance to chemotherapeutic or radiation therapy. Accordingly, the invention provides for co-administration of therapeutically effective amounts of one or more compound of the invention, e.g., Nrf2 inhibitors, in combination with one or more additional cancer therapeutic, e.g,. a chemotherapeutic.

The term “therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition

The effect of a combination treatment of the present invention is expected to be a synergistic effect. According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synerg istic if the effect is therapeutically superior to the effect achievable with either compound or treatment alone. Further, the effect of the combination treatment is synergistic if a beneficial effect is obtained in a group of patients that does not respond (or responds poorly) to a particular treatment alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of a standard chemotherapeutic or radiation treatment may be reduced without detriment to one or more of the extent of the response, the response rate, the time to disease progression and survival data, in particular without detriment to the duration of the response, but with fewer and/or less troublesome side effects than those that occur when conventional doses of each component are used.

Chemotherapeutic agents for optional use with the combination treatment of the present invention may include, for example, the following categories of therapeutic agent:

(i) antiproliferative/antineoplastic drugs and combinations thereof as used in medical oncology (for example carboplatin and cisplatin);

(ii) cytostatic agents, for example inhibitors of growth factor function such as growth factor antibodies, growth factor receptor antibodies (for example the anti-erbB2 antibody trastuzumab and the anti-erbB1 antibody cetuximab), Class I receptor tyrosine kinase inhibitors (for example inhibitors of the epidermal growth factor family), Class II receptor tyrosine kinase inhibitors (for example inhibitors of the insulin growth factor family such as IGF1 receptor inhibitors as described, for example, by Chakravarti et al., Cancer Research, 2002, 62: 200-207), serine/threonine kinase inhibitors, farnesyl transferase inhibitors and platelet-derived growth factor inhibitors;

(iii) antiangiogenic agents such as those which inhibit the effects of vascular endothelial growth factor (for example the anti-vascular endothelial cell growth factor antibody bevacizumab and VEGF receptor tyrosine kinase inhibitors such as 4-(4-bromo-2-fluoroanilino)-6-methoxy-7-(1-methylpiperidin-4-ylme-thoxy)quinazoline (ZD6474; Example 2 within WO 01/32651), 4-(4-fluoro-2-methylindol-5-yloxy)-6-methoxy-7-(3-pyrrolidin-1-ylpropoxy)-quinazoline (AZD2171; within WO 00/47212), vatalanib (PTK787; WO 98/35985) and SU11248 (WO 01/60814));

(iv) vascular damaging agents such as the compounds disclosed in International Patent Applications WO 99/02166, WO 00/40529, WO 00/41669, WO 01/92224, WO 02/04434 and WO 02/08213;

(v) biological response modifiers (for example interferon); and

(vi) a bisphosphonate such as tiludronic acid, ibandronic acid, incadronic acid, risedronic acid, zoledronic acid, clodronic acid, neridronic acid, pamidronic acid and alendronic acid.

Specific anti-cancer chemotherapeutics include the following:

Anti-cancer or anti-cell proliferation agents including, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hotmone (GNRH) analogues, progestrins, immunomodulators, miscellaneous antineoplastics, photosensitizing agents, and skin & mucous membrane agents. See, Physician's Desk Reference, 2001 Edition.

Adjunct antineoplastic agents including Anzemet® (Hoeschst Marion Roussel), Aredia® (Novartis), Decadron® (Merck), Deltasone® (Pharmacia), Didronel® (MGI), Diflucan® (Pfizer), Epogen® (Amgen), Ergamisol® (Janssen), Ethyol® (Alza), Kenacort® (Bristol-Myers Squibb), Kytril® (SmithKline Beecham), Leucovorin® (Immunex), Leucovorin® (Glaxo Wellcome), Leucovorin® (Astra), Leukine® (Immunex), Marinol® (Roxane), Mesnex® (Bristol-Myers Squibb Oncology/Immunology, Neupogen (Amgen), Procrit® (Ortho Biotech), Salagen® (MGI), Sandostatin® (Novartis), Zinecard® (Pharmacia & Upjohn), Zofran® (Glaxo Wellcome) and Zyloprim® (Glaxo Wellcome).

Alkylating agents including Myleran® (Glaxo Wellcome), Paraplatin® (Bristol-Myers Squibb Oncology/Immunology), Platinol® (Bristol-Myers Squibb Oncology/Immunology), and Thioplex® (Immunex).

Nitrogen mustards including Alkeran® (Glaxo Wellcome), Cytoxan® (Bristol-Myers Squibb Oncology/Immunology), Ifex® (Bristol-Myers Squibb Oncology/Immunology), Leukeran® (Glaxo Wellcome) and Mustargen® (Merck).

Nitrosoureas including BiCNU® (Bristol-Myers Squibb Oncology/Immunology), CeeNU® (Bristol-Myers Squibb Oncology/Immunology), Gliadel® (Rhone-Poulenc Rover) and Zanosar® (Pharmacia & Upjohn).

Antibiotics including Adriamycin PFS/RDF® (Pharmacia & Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia & Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology).

Antimetabolites including Cytostar-U® (Pharmacia & Upjohn), Fludara® (Berlex), Sterile FUDR® (Roche Laboratories), Leustatin® (Ortho Biotech), Methotrexate® (Immunex), Parinethol® (Glaxo Wellcome), Thioguanine® (Glaxo Wellcome) and Xeloda® (Roche Laboratories).

Androgens including Nilandron® (Hoechst Marion Roussel) and Teslac® (Bristol-Myers Squibb Oncology/Immunology).

Antiandrogens including Casodex® (Zeneca) and Eulexin® (Schering).

Antiestrogens including Arimidex® (Zeneca), Fareston® (Schering), Femara® (Novartis) and Nolvadex® (Zeneca). Suitable estrogens including Estrace® (Bristol-Myers Squibb) and Estrab® (Solvay).

Gonadotropin releasing hormone (GNRH) analogues include Leupron Depot® (TAP) and Zoladex® (Zeneca).

Progestins including Depo-Provera® (Pharmacia & Upjohn) and Megace® (Bristol-Myers Squibb Oncology/Immunology)

Immunomodulators including Erganisol® (Janssen), Proleukin® (Chiron Corporation), Thalomid® (Celgene Corporation), Revlimid® (Celgene Corporation) and Tetra-hydro-biopterine.

Antineoplastics including Camptosar® (Pharmacia & Upjohn), Celestone® (Schering), DTIC-Dome® (Bayer), Elspar® (Merck), Etopophos® (Bristol-Myers Squibb Oncology/Immunology), Etopoxide® (Astra), Gemzar® (Lilly), Hexalen® (U.S. Bioscience), Hycantin® (SmithKline Beecham), Hydrea® (Bristol-Myers Squibb Oncology/Immunology), Hydroxyurea® (Roxane), Intron A® (Schering), Lysodren® (Bristol-Myers Squibb Oncology/Immunology), Navelbine® (Glaxo Wellcome), Oncaspar® (Rhone-Poulenc Rover), Oncovin® (Lilly), Proleukin® (Chiron Corporation), Rituxan® (IDEC), Rituxan® (Genentech), Roferon-A® (Roche Laboratories), Taxol® (Bristol-Myers Squibb Oncology/Immunology), Taxotere® (Rhone-Poulenc Rover), TheraCys® (Pasteur Merieux Connaught), Tice BCG® (Organon), Velban® (Lilly), VePesid® (Bristol-Myers Squibb Oncology/Immunology), Vesanoid® (Roche Laboratories), Vumon® (Bristol-Myers Squibb Oncology/Immunology) and Nicotinamide.

Radiotherapy may be administered according to the known practices in clinical radiotherapy. The dosages of ionising radiation will be those known for use in clinical radiotherapy. The radiation therapy used will include for example the use of y-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0Gy, 5 days a week for 5-6 weeks.

Normally a total fractionated dose will lie in the range 45-60Gy. Single larger doses, for example 5-10Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

In some embodiments, an Nrf2 inhibitor can be co-administered with other therapeutics and/or part of a treatment regimen that includes radiation therapy.

The co-administration of therapeutics can be sequential in either order or simultaneous. In some embodiments an Nrf2 inhibitor is co-administered with more than one additional therapeutic.

The therapeutic regimens can include sequential administration of a Nrf2 inhibitor and initiation of radiation therapy in either order or simultaneously. Those skilled in the art can readily formulate an appropriate radiotherapeutic regimen. Carlos A Perez & Luther W Brady: Principles and Practice of Radiation Oncology, 2nd Ed. JB Lippincott Co, Phila., 1992, which is incorporated herein by reference describes radiation therapy protocols and parameters which can be used in the present invention.

When used in as part of the combination therapy the therapeutically effective amount of the inhibitor may be adjusted such that the amount is less than the dosage required to be effective if used without other therapeutic procedures.

In some preferred embodiments, treatment with pharmaceutical compositions according to the invention is preceded by surgical intervention.

According to the present invention, methods of treating cancer in individuals who have been identified as having cancer are performed by delivering to such individuals an amount of a Nrf2 inhibitor sufficient to induce apoptosis in tumor cells in the individual. By doing so, the tumor cells will undergo apoptosis and the tumor itself will reduce in size or be eliminated entirely. Thus, Patient survival may be extended and/or quality of life improved as compared to treatment that does not include Nrf2 inhibitor.

The pharmaceutical compositions described above may be administered by any means that enables the active agent to reach the agent's site of action in the body of the individual. The dosage administered varies depending upon factors such as: pharmacodynamic characteristics; its mode and route of administration; age, health, and weight of the recipient; nature and extent of symptoms; kind of concurrent treatment; and frequency of treatment.

The amount of compound administered will be dependent on the subject being treated, on the subject's weight, the severity of the affliction, the manner of administration and the judgment of the prescribing physician. In some embodiments, the dosage range would be from about 1 to 3000 mg, in particular about 10 to 1000 mg or about 25 to 500 mg, of active ingredient, in some embodiments 1 to 4 times per day, for an average (70 kg) human. Generally, activity of individual compounds used in the invention will vary.

Dosage amount and interval may be adjusted individually to provide plasma levels of the compounds which are sufficient to maintain therapeutic effect. Usually, a dosage of the active ingredient can be about 1 microgram to 100 milligrams per kilogram of body weight. In some embodiments a dosage is 0.05 mg to about 200 mg per kilogram of body weight. In another embodiment, the effective dose is a dose sufficient to deliver from about 0.5 mg to about 50 mg. Ordinarily 0.01 to 50 milligrams, and in some embodiments 0.1 to 20 milligrams per kilogram per day given in divided doses 1 to 6 times a day or in sustained release form is effective to obtain desired results. In some embodiments, patient dosages for administration by injection range from about 0.1 to 5 mg/kg/day, preferably from about 0.5 to 1 mg/kg/day. Therapeutically effective serum levels may be achieved by administering multiple doses each day. Treatment for extended periods of time will be recognized to be necessary for effective treatment.

In some embodiments, the route may be by oral administration or by intravenous infusion. Oral doses generally range from about 0.05 to 100 mg/kg, daily. Some compounds used in the invention may be orally dosed in the range of about 0.05 to about 50 mg/kg daily, while others may be dosed at 0.05 to about 20 mg/kg daily.

The invention futher provides kits comprising one of more Nrf2 inhibitors and instructions for use in treating cancer. The kit may furhte comprise one or more additional anticancer treatments.

Suitable cancers that can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma;

Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyo sarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In further embodiments, the invention provides diagnostic methods for determining if a subject will become, or has an increased chance of becoming, resistant to readiation or chemotherapeutic treatment.

### EXAMPLE 1

### NRF2 Regulates Drug resistance and Cancer Progression

This example demonstrates that loss of function mutations in the NRF2 inhibitor, Kelch-like ECH-associated protein (KEAP1) results in gain of NRF2 function in non-small-cell lung cancer (NSCLC). Using RNAi approach, this example demonstrates that gain of NRF2 function in lung cancer cells promotes tumorigenicity and contributes to chemo- and radioresistance by upregulation of glutathione, thioredoxin and the drug efflux pathways involved in detoxification of a broad spectrum of drugs and electrophiles. Inhibiting NRF2 expression in human lung tumors using naked siRNA duplexes in combination with carboplatin and radiation significantly inhibits tumor growth in both a subcutaneous model and an orthotopic model of lung cancer.

KEAP1 constitutively suppresses NRF2 activity in the absence of stress. Oxidants, xenobiotics and electrophiles hamper the KEAP1-mediated proteasomal degradation of NRF2, which results in increased nuclear accumulation and transcriptional induction of target genes. The NRF2-regulated transcriptional program includes a broad spectrum of genes, including genes encoding antioxidants (e.g., the glutathione system: y-glutamyl cysteine synthetase modifier subunit [GCLm], γ-glutamyl cysteine synthetase catalytic subunit [GCLc], glutathione synthetase [GSS], glutathione reductase [GSR], glutathione peroxidase [GPX] and the cysteine/glutamate transporter [SLC7A11] which transports cysteine for synthesis of glutathione); the thioredoxin system: thioredoxin-1 [TXN], thioredoxin reductase [TXNRD1] and peroxiredoxins [PRDX], xenobiotic metabolism enzymes (e.g., NADP[H] quinone oxidoreductase 1 [NQO1], UDP-glucuronosyltransferase) and members of the glutathione-S-transferase family [GSTs]), and several ATP-dependent multidrug resistant drug efflux pumps (e.g., ABCC1 and ABCC2) (Hayashi et al., 2003; Kim et al., 2007; Lee et al., 2005; Nguyen et al., 2003; Rangasamy et al., 2004; Rangasamy et al., 2005; Thimmulappa et al., 2006; Vollrath et al., 2006). NRF2 also protects against apoptosis induced by oxidants and FAS ligand (Kotlo et al., 2003; Morito et al., 2003; Rangasamy et al., 2004). Downregulation of NRF2 using anti-sense RNA resulted in cell sensitization to apoptosis (Kotlo et al., 2003). Thus, NRF2 promotes survival against stress caused by exposure to radiation, electrophiles and xenobiotics.

### Experimental Procedures

Cell Culture and Reagents: A549 and H460 cells were purchased from American Type Culture Collection (Manassas, Va., United States) and cultured under recommended conditions. All transfections were carried out using Lipofectamine 2000 (Invitrogen, CA). A549 cells stably expressing luciferase (A549-luc-C8 cells) were purchased from Xenogen corporation, CA. A549 cells stably expressing antioxidant response element (ARE) reporter was generated as described earlier (Singh et al., 2006a).

Generation of lung cancer cell lines stably expressing NRF2 shRNA: To inhibit the expression of NRF2, we designed a short hairpin RNA targeting the 3′ end of the NRF2 transcript as described in our previous reports (Singh et al., 2006a; Singh et al., 2006b). The NRF2 shRNA duplex with the following sense and antisense sequences was used: 5′-GATCC GTAAGAAGCCAGATGTTAATTCAAGAGACATTCTTCGGTCTACAATTTTTTTTGGAA A-3′ (sense) (SEQ ID NO:7) and 5′-AGCTTTTCCAAAAAAAATTGTAGACCGAAGAATG TCTCTTGAA TTAACATCTGGCTTCTTAC G-3′ (antisense) (SEQ ID NO:8) (Singh et al., 2006a). Short hairpin RNA cassette was subcloned into pSilencer vector and transfected into

A549 and H460 cells. A short hairpin RNA targeting luciferase gene was used as control. Stable cell clones with reduced NRF2 expression were generated. We screened 15 clones transfected with NRF2 shRNA and 10 clones transfected with Luc-shRNA for each cell line. All the clones were screened by real time quantitative PCR and immunoblotting.

For the in vivo experiments, all siRNA compounds were chemically synthesized being stabilized by 20′-Me modifications (Biospring, Frankfurt, Germany). The sequence of siRNA targeting human NRF2 used for in vivo experiments is 5′-UCCCGUUUGUAGAUGACAA-3′ (sense) (SEQ ID NO:9) and 5′-UUGUCAUCUACAAACGGGA-3′ (antisense) (SEQ ID NO:10). The sequence of control siRNA targeting GFP is 5′-GGCUACGUCCAGGAGCGCACC-3′ (SEQ ID NO:11) (sense) and 5′-GGUGCGCUCCUGGACGUAGC-3′ (antisense) (SEQ ID NO:12) (Hamar et al., 2004).

Real Time RT-PCR: Total RNA was extracted from lung tumors and or cells using the RNeasy kit (Qiagen) and was quantified by UV absorbance spectrophotometry. The reverse transcription reaction was performed by using the Superscript First Strand Synthesis system (Invitrogen) in a final volume of 20 μl containing 2 μg of total RNA, 100 ng of random hexamers, 1×reverse transcription buffer, 2.5 mM MgCl₂, 1 mM dNTP, 10 units of RNaseOUT, 20 units of Superscript reverse transcriptase, and nuclease free water. Quantitative real time RT-PCR analyses of Human NRF2, GCLc, GCLm, GSR, xCT, G6PDH, PRDX1, GSTM4, MGST1, NQO1, HO-1, TXN1, TXNRD1, ABCC1, and ABCC2 were performed by using assay on demand primers and probe sets from Applied Biosystems. Assays were performed using the ABI 7000 Taqman system (Applied Biosystems). β-ACTINwas used for normalization.

Western Blot Analysis: To obtain total protein lysates, cancer cells were lysed in 50 mM Tris (pH 7.2), 1% Triton X-100 containing Halt Protease Inhibitor cocktail (Pierce, Rockford, Ill., United States) and centrifuged at 12,000 g for 15 min at 4° C. For immunoblot analysis, 100 μg of total protein lysate was resolved on 10% SDS-PAGE gels. Proteins were transferred onto PVDF membranes, and the following antibodies were used for immunoblotting: anti-NRF2, anti-TXNRD1 and anti-actin (H-300; Santa Cruz Biotechnology, Santa Cruz, Calif., United States), anti-GAPDH (Imgenex, Sorrento Valley, Calif., United States) and anti-TXN (American Diagnostica, Greenwich, Conn., USA). All primary antibodies were diluted in PBS-T/5% nonfat dry milk and incubated overnight at 4° C.

Clonogenic assays: Exponentially growing cells were counted, diluted and seeded in triplicate at 1000 cells per culture dish (100 mm). Cells were incubated for 24 h in a humidified CO₂ incubator at 37° C., exposed to high dose rate (0.68Gy/min) radiation using a Gamma cell 40 ¹³⁷Cs irradiator (Atomic Energy of Canada, Ltd). To assess clonogenic survival following radiation exposure, cell cultures were incubated in complete growth medium at 37° C. for 14 days and then stained with 50% methanol-crystal violet solution. Only colonies with more than 50 cells were counted, and the surviving fraction was calculated and compared to the control.

Measurement of ROS levels: Cells were incubated with 10 μM c-H₂DCFDA (molecular probes, Invitrogen, CA) for 30 mins at 37° C. to assess the ROS mediated oxidation to the fluorescent compound c-H2DCF. Fluorescence of oxidized c-H₂DCF was measured at an excitation wavelength of 480 nM and an emission wavelength of 525 nM using a FAC Scan flow cytometer (Becton Dickinson).

Enzyme Assay: Enzyme activities of GST, GSR and NQO1 and G6PDH were determined in the total protein lysates by following methods previously described (Thimmulappa et al., 2002).

Drug Accumulation Assay: A549-NRF2shRNA and H460-NRF2shRNA cells as well as their respective control cells expressing Luc-shRNA were seeded at a density of 0.3×10⁶ cells/ml in 6-well plates. After 12 h, growth medium was aspirated and replaced with 1.5 ml of RPMI 1640 containing 0.2 μM of [³H] Etoposide (646 mCi/mmol; Moravek Biochemicals) and [¹⁴C] Carboplatin (53 mCi/mmol; Amersham Biosciences). Cells were incubated with radiolabeled drug for indicated period of time and then cooled on ice, washed four times with ice-cold PBS, and solubilized with 1.0 ml of 1% SDS. The radioactivity in each sample was determined by scintillation counting. Results are presented as means±SD. Comparisons were made by paired t-test and P<0.05 was considered statistically significant.

MTS Cell Viability Assay: The in vitro drug sensitivity to etoposide and carboplatin was assessed using Cell Titer 96 Aqueous assay kit (Promega). Cells were plated at a density of 5,000 cells/well in 96-well plates. They were allowed to recover for 12 h and then exposed to various concentrations of etoposide and carboplatin for 72-96 h. Drug cytotoxicity was evaluated by adding 40 μl of 3-(4,5-dimethylthiazol-2-yl)-5-)3-Carboxymethoxyphenyl)-2-(sulfophenyl)-2H-tetrazolium solution. The plates were incubated at 37° C. for two and absorbance at 490 nM was measured. Each combination of cell line and drug concentration was set up in eight replicate wells, and the experiment was repeated three times. Each data point represents a mean±SD and normalized to the value of the corresponding control cells.

Cell Proliferation assay: Cellular proliferation was analyzed using the colorimetric MTS assay (Promega). Briefly, H460 cells (1000 cells/well) and A549 cells (1500 cells/well) were plated in 96-well plates and the growth rate was measured.

Soft agar growth assay: A549 and H460 cells (2×10⁴) stably expressing NRF2 shRNA or the control Luc-shRNA were diluted in 4 ml of DMEM medium containing 10% serum and 0.4% low melting point (LMP) agarose. This mixture was subsequently placed over 5 ml of hardened DMEM medium containing 10% serum and 1% LMP and allowed to harden at room temperature. The cells were allowed to grow for 2-3 weeks, after which visible colonies containing greater than 50 cells were counted.

Tumor Xenografts and siRNA Treatment: We injected A549 cells (5×10⁶) and H460 cells (2×10⁶) subcutaneously into the hind leg of male athymic nude mice and measured the tumor dimensions by caliper once per week. The tumor volumes were calculated using the following formula: [length (mm)×width (mm)×width (mm)×0.52]. For in vivo delivery of siRNA into tumors, siRNA duplexes diluted in PBS were injected into the tumors using insulin syringes at a concentration of 10 μg of siRNA/50 mm³ of tumor volume. Intraperitoneal injections of carboplatin were given at a dose of 40 mg/kg body weight. Both siRNA and carboplatin were administered twice weekly for 4 weeks. Upon termination, tumors were harvested and weighted. For radiation exposure, mice with subcutaneous tumors were exposed to high dose rate radiation (2 dose of 3Gy each) using a Gamma cell 40 ¹³⁷Cs irradiator (Atomic Energy of Canada, Ltd).

Experimental Lung Metastasis: In experimental metastasis experiments, 2×10⁶ A549-C8-luc cells were injected into SCID-Beige mice (Charles River, Mass.) intravenously. For delivery of siRNA into lung tumors, 100 μg of siRNA duplex diluted in PBS was aerosolized using a nebulizer. Mice were given three doses of siRNA (100 μg/dose) every week, for 4 weeks, using a nebulizer. Intraperitoneal injections of carboplatin were given at a dose of 30 mg/kg body weight twice/week. All experimental animal protocols were performed in accordance with guidelines approved by the animal care committee at the Johns Hopkins University Bloomberg School of Public Health.

In Vivo Imaging: For luminescent imaging, animals inoculated with A549-C8-luc cells, which express a luciferase reporter gene, were anesthetized and injected intraperitoneally with 250 ul of luminescent substrate (15 mg/ml stock) D-Luciferin Firefly (Xenogen Cat# XR-1001). The animals were then imaged and analyzed by using the Xenogen IVIS Optical Imaging Device in the Johns Hopkins Oncology Center.

siRNA Delivery into Lung Tumors: Female C57B6 mice were injected with Lewis Lung Carcinoma (LLC) cells (0.5×10⁶) intravenously, 24 days prior to the delivery experiment. Upon development of lung metastases, mice were administered with 100 μg/mouse of Cy3-labeled naked chemically stabilized reference siRNA via nebulizer inhalation on 3 consequent days. Mice were euthanized 24 hrs after the last inhalation. Upon termination, lungs were inflated with ice-cold 4% paraformaldehyde, followed by manual sectioning with razor blades. Clearly visible large surface tumors were sectioned separately. Resulting sections were analyzed by Bio-Rad Confocal microscope using a 20×Water objective and 2×zoom combined to give a total of 40×magnification. Control, non-siRNA-treated lungs were used to set up background fluorescence level.

Statistical Analysis—Statistical comparisons were performed by Student's t-tests or Wilcoxon rank-sum test. A value of p<0.05 was considered statistically significant. Tumor weights and changes in tumor volume were summarized using descriptive statistics. Differences in tumor measures between treatment groups were examined using linear regression models with generalized estimating equations (GEE). The distributions of both tumor measurements were skewed, so log transformations were used.

### Results

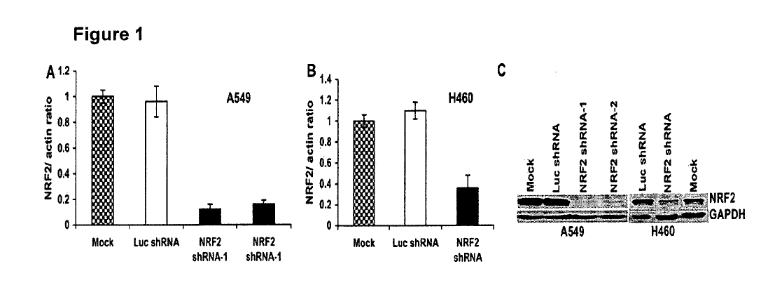

Generation of lung cancer cell lines stably expressing NRF2shRNA: To inhibit the expression of NRF2, we designed a short hairpin RNA targeting the 3′ end of the NRF2 transcript as described in our previous reports (Singh et al., 2006a; Singh et al., 2006b). Short hairpin RNA cassette was subcloned into pSilencer vector and transfected into A549 and H460 cells. A short hairpin RNA targeting luciferase gene was used as control. Stable cell clones with reduced NRF2 expression were generated. We screened 15 clones transfected with NRF2 shRNA and 10 clones transfected with luciferase shRNA for each cell line. All the clones were screened by real time quantitative PCR and immunoblotting. After initial screening, we selected two independent clones of A549 cells expressing NRF2 shRNA, which demonstrated a stable 85% downregulation of NRF2 mRNA (FIG. 1A). A single clone expressing NRF2 shRNA derived from H460 cells demonstrated 70% inhibition of NRF2 mRNA (FIG. 1B). Measurement of NRF2 protein by western blotting showed similar decrease in protein levels (FIG. 1C). The expression of NRF2 did not change between the control cells transfected with luciferase shRNA and the untransfected cancer cells (FIG. 1C).

Lowering NRF2 expression in A549 and H460 cells causes global decrease in expression of electrophile and drug detoxification system. Lowering of NRF2 levels leads to a decline in the expression of electrophile and drug detoxification genes in normal cells. The expression of selected electrophile and drug detoxification genes were determined in two clones of A549 and one clone of H460 cells stably expressing NRF2 shRNA using real time RT-PCR (Table 1).

Lowering NRF2 level by RNAi in the A549 and H460 cells decreased the mRNA expression of the genes that constitute the glutathione system (γ-glutamyl cysteine synthetase modifier subunit (GCLM), γ-glutamyl cysteine synthetase catalytic subunit (GCLC), glutathione reductase (GSR), and the cysteine/glutamate transporter (SLC7A11) that transports cysteine for synthesis of glutathione) as well as the glutathione-dependent enzymes Glutathione peroxidase 2 (GPx2), Glutathione peroxidase 3 (GPx3) and Glutathione S-transferase's (MGST1 and GSTM4) (Table 1).

Enzyme activity measurements for selected gene products (GSR, GPX and GST) were carried out to determine the extent to which their transcriptional inhibition paralleled changes in their activities. There was significant decrease in activities of all of these enzymes in the A549-NRF2shRNA and H460-NRF2shRNA cells relative to the cells expressing luciferase shRNA (FIG. 12). Direct measurement of intracellular GSH concentration by Teitz assay demonstrated a decrease in GSH levels by ,,50% in A549 cells and ,,30% in H460 cells expressing NRF2 shRNA (Supplementary FIG. S1).

Lowering of NRF2 in A549 and H460 cell caused significant decreases in the mRNA for TXN and TXNRD1 that constitute the thioredoxin system which has been associated with therapeutic resistance (Table 1). Protein levels of TXN and TXNRD1 did not change between control A549 cells expressing luciferase shRNA and the untransfected cells (Supplementary FIG. S1).

NADPH is required to provide reducing equivalents for the regeneration of reduced glutathione and thioredoxin by GSR and TXNRD1. Expression of genes encoding the NADPH biosynthesis enzymes, such as glucose-6-phosphate dehydrogenase (G6PDH) and malic enzyme 1 (ME1) were downregulated in the A549-NRF2shRNA and H460-NRF2shRNA cells suggesting the dependence of these genes on NRF2 for their expression (Table 1). Consistent with low transcript levels, G6PDH enzyme activity was significantly downregulated in A549-NRF2shRNA and H460-NRF2shRNA (Supplementary FIG. S1).

We also found that other antioxidant genes such as NAD(P)H dehydrogenase, quinine 1 (NQO1), heme oxygenase-1 (HO-1) and peroxiredoxin 1 (PRDX1) were downregulated as a result of lowering of NRF2 by shRNA in cancer cells (Table 1). Furthermore, the transcript levels of multidrug resistance protein like ATP-binding cassette, sub family C, member 1 (ABCC1) and ATP-binding cassette, sub family C, member 2 (ABCC2), were significantly downregulated in cells expressing NRF2 shRNA. Thus, downregulation of NRF2 profoundly decreased the expression of antioxidant enzymes and electrophile and drug detoxification systems in cancer cells with gain of NRF2 function.

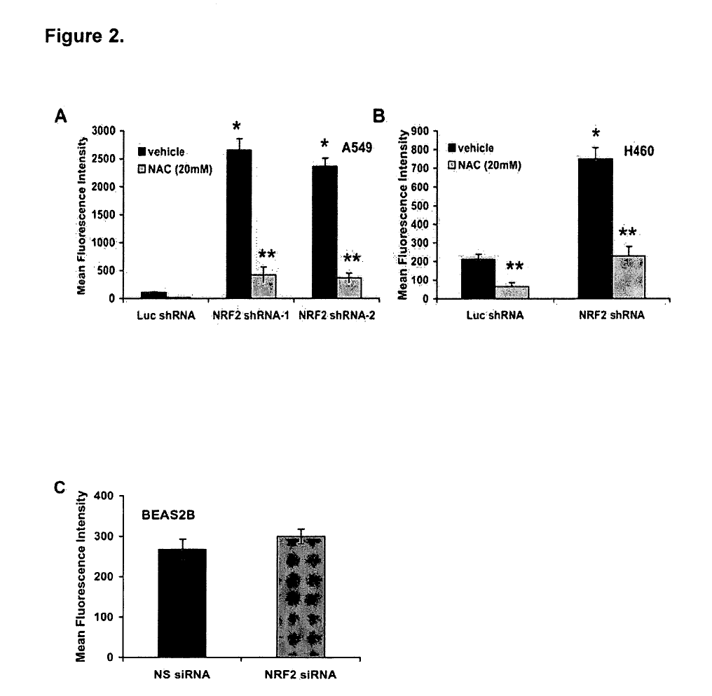

Enhanced production of ROS in cells stably transfected with NRF2 shRNA: To determine the degree of overall increase in oxidative stress as a result of global decrease in the expression of electrophile detoxification system by downregulating NRF2, intracellular ROS levels were monitored using 2′, 7′-dichlorodihydrofluorescein diacetate (c-H₂DCFDA) and flow cytometry. Oxidation of c-H₂DCFDA leads to an increase in the fluorescent product dichlorodihydrofluorescein, permitting the quantification of relative levels of ROS. The results demonstrated an increase in fluorescence in both A549-NRF2shRNA and H460-NRF2shRNA cells (FIG. 2A-B). A549-NRF2 shRNA cells demonstrated a pronounced 25-fold increase in ROS level where as H460-NRF2shRNA cells demonstrated a 3.5-fold increase in ROS levels. Treatment of these cells with non specific radical scavenger NAC for 30 mins reduced ROS production and attenuated the mean fluorescent intensity in A549-NRF2shRNA and H460-NRF2shRNA by 85% and 75% in A549 and H460 cells respectively. These results suggest that the generation of ROS at a steady state is relatively increased in NRF2 shRNA transfectants than in control Luc-shRNA cells. Interestingly, inhibition of NRF2 activity in non-tumorigenic BEAS2B cells did not show a significant increase in ROS (FIG. 2C). Thus, constitutive NRF2 activity is indispensable for maintaining redox balance in cancer cells unlike normal cells in the absence of stress.

Decrease in NRF2 expression by shRNA leads to increased drug accumulation and enhanced chemosensitivity in cancer cells: Since NRF2 shRNA causes decrease in expression of drug detoxification enzymes as well as drug efflux pumps, we measured drug accumulation in cancer cells (H460 and A549) stably transfected with shRNA targeting NRF2. A non-specific shRNA targeting luciferase (Luc shRNA) was used as control. To analyze drug accumulation, cells were incubated with radiolabeled drug and intracellular drug content was assayed at various time points. The amount of drug accumulation was substantially increased in NRF2 shRNA at 60 mins and 120 mins. The NRF2 shRNA transfectants accumulated 2-3 fold more drug than the control cells at 60 mins and the difference in drug content remained same or increased at 120 mins. Increased drug accumulation in cells with low levels of NRF2 protein suggests that NRF2 plays an important role in regulating the accumulation of drug in the cancer cells (FIG. 13)

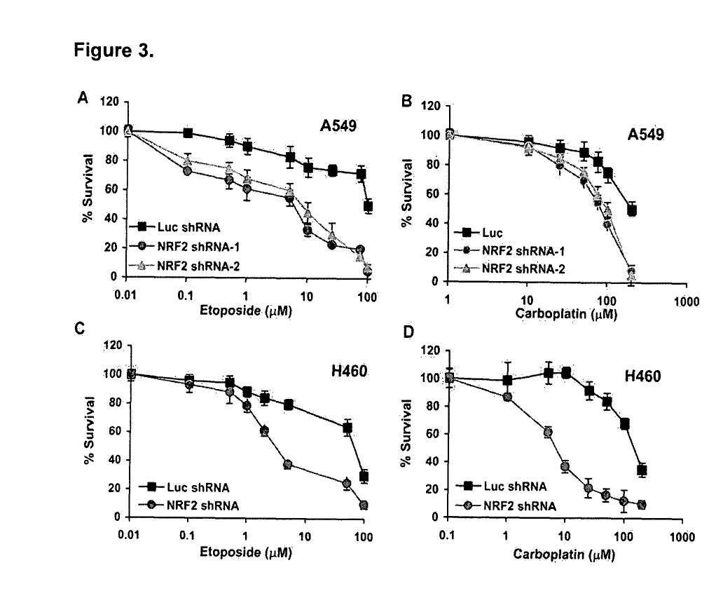

To study whether targeting NRF2 expression enhances the sensitivity of lung cancer cells to chemotherapeutic drugs like carboplatin and etoposide, we used A549 and H460 cells stably expressing NRF2 shRNA. We treated these cell populations with escalating concentrations of carboplatin and etoposide. The concentrations were selected after pilot experiments to determine the maximum amount of drug that revealed survival differences between A549 and H460 cells expressing control shRNA and its derivatives expressing anti-NRF2 shRNA. We found that lowering of NRF2 expression in both A549 and H460 cell lines greatly enhanced the cytotoxicity (,,30-70%) of these drugs resulting in increased cell death compared to the control shRNA group (FIG. 3A-D). The IC₅₀ doses of carboplatin and etoposide was followed by a reduction in the number of viable cells to 50% as compared with vehicle treated control cells. The IC₅₀ for carboplatin and etoposide decreased in both A549-NRF2shRNA and H460-NRF2shRNA cells when compared with their respective control cells expressing luciferase shRNA.

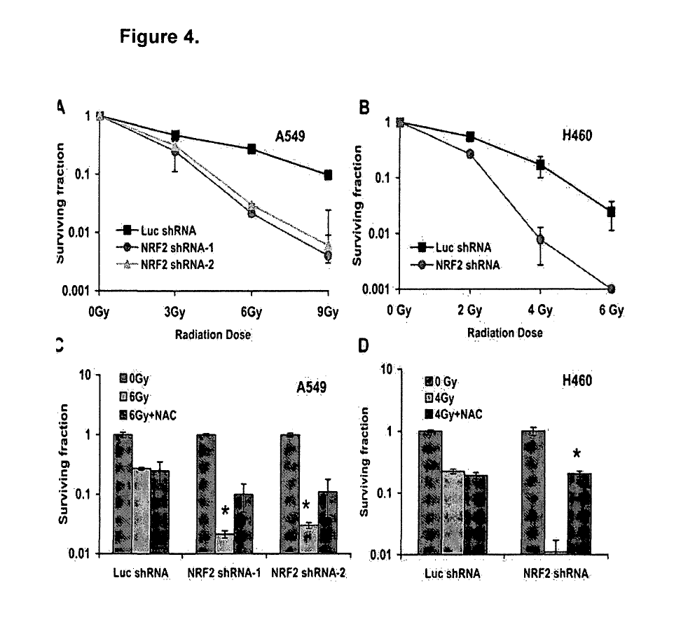

Downregulation of NRF2 causes radiosensitization: Next, we determined whether inhibition of NRF2 expression, which causes a decrease in the electrophile detoxification system, could also alter cellular responses to ionizing radiation. A549 and H460 cells stably expressing NRF2 shRNA and control non targeting shRNA transfectants were exposed to ionizing radiation, then assayed for in vitro cell clonogenic survival. Clonogenic survival in all the cell lines decreased as the radiation dose increased, as expected. The NRF2 shRNA transfectants showed a markedly increased radiosensitivity that was more pronounced at higher doses, as compared with cells transfected with the non targeting control shRNA. Thus, attenuation of NRF2 activity by shRNA enhanced radiosensitivity in both A549 and H460 cells (FIG. 4A-B). At a dose of 6 Gy, the surviving fraction of the A549-NRF2shRNA cells was approximately 2-3% compared with 27% for the A549-Luc shRNA cells. Similarly, a dose of 4Gy to H460-NRF2shRNA cells reduced the survival to ,,0.7% relative to 20% for the H460-Luc shRNA cells. There was no significant difference in radiosensitivity between the control non-targeting shRNA-transfected and the parental lung cancer cell lines (data not shown). We further examined whether blockade of ROS generation in cells expressing NRF2 shRNA by NAC pretreatment could reverse the increased sensitivity to ionizing radiation (FIG. 4C-D). The cell clonogenic survival assay showed that decreasing the spike in ROS, as a result of downregulation of NRF2, rescued cell death as demonstrated from increased number of colonies in the clonogenic assay. These results clearly indicate that downregulation of NRF2 causes radiosensitization in a ROS-dependent manner.

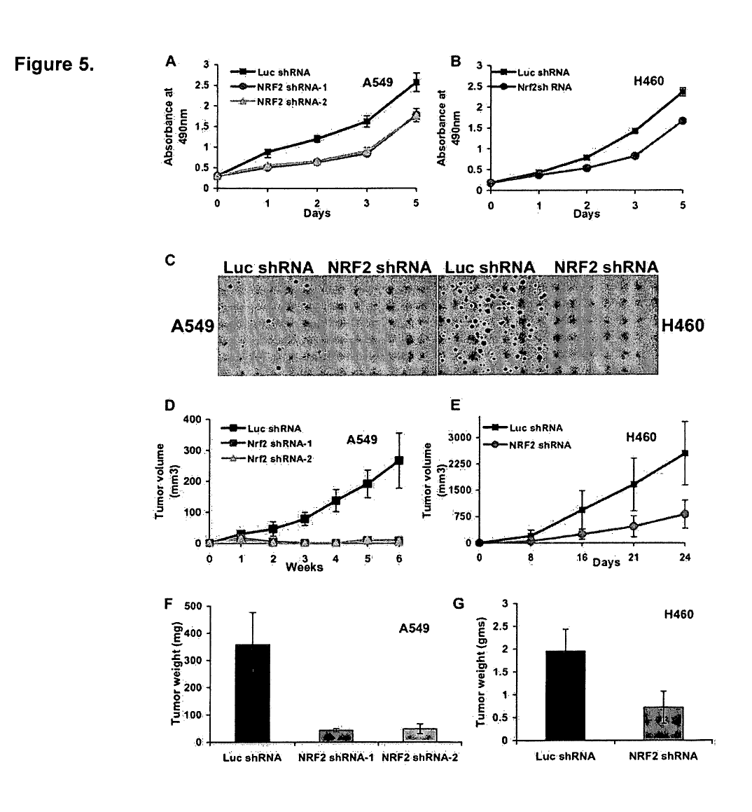

NRF2 is required for anchorage independent growth and tumor formation in vivo. The misexpression of NRF2 prompted us to examine its significance in the tumorigenic properties in the non small cell lung cancer cells. Depletion of NRF2 in both the cancer cell lines resulted in a pronounced decrease in cellular proliferation as measured by MTS assay (FIG. 5A-B). We also determined the ability of A549-NRF2shRNA and H460-NRF2shRNA cells to form colonies in soft agar. Suppression of NRF2 in H460 and A549 cell lines resulted in a substantial reduction on colony formation in soft agar compared to the control cells expressing luciferase shRNA (FIG. 5C). In order to further examine the affect of NRF2 suppression on lung tumorigenesis, we injected A549-NRF2shRNA and H460-NRF2shRNA and their corresponding control cells expressing Luc-shRNA into the flank of athymic nude mice and monitored the increase in tumor volume over a 4-6 week period. Weight of the tumor was recorded at the termination of the experiment. Significantly, suppression of NRF2 in the A549 cells resulted in complete inhibition of tumor formation whereas H460-NRF2shRNA cells showed a less dramatic yet significant and reproducible reduction in tumor volume (FIG. 5D-E). Mean difference in tumor weight between the Luc-shRNA and NRF2 shRNA expressing H460 cells was 1.24 gms (95% CI=0.773 to 1.71; P=0.0001) (FIG. 5F-G). Data was analyzed using two-sample Wilcoxon rank-sum (Mann-Whitney) test. These data indicate that NRF2 is required for maintenance of the transformed phenotype in vitro and in vivo.

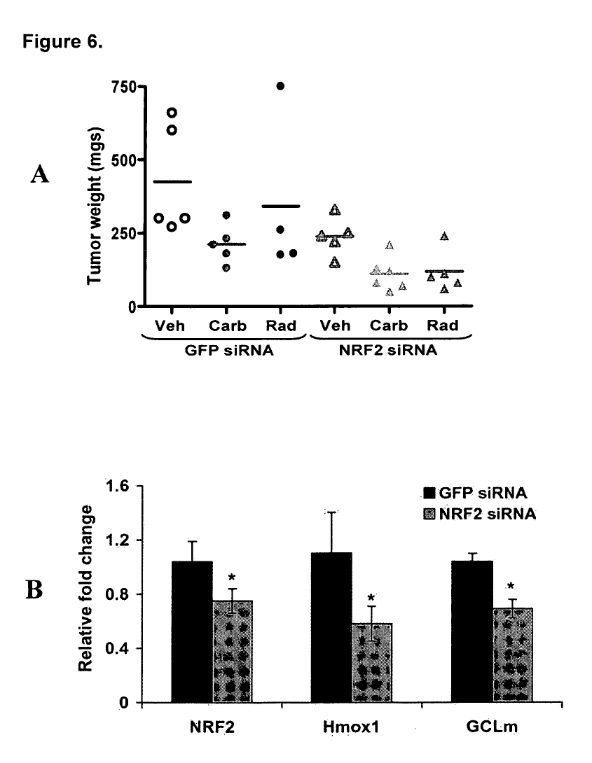

Therapeutic efficacy of NRF2 siRNA in combination with carboplatin and radiation in vivo: To elucidate whether the synergistic mode of action of NRF2 shRNA and carboplatin observed in cell culture occurs in vivo, we performed a xenograft experiment with A549 cells. Mice bearing subcutaneous tumors were randomly allocated to one of the following groups with therapy beginning 15 days after tumor cell injection: GFP siRNA, GFP siRNA+carboplatin, GFP siRNA+radiation, NRF2 siRNA, NRF2 siRNA+carboplatin and NRF2 siRNA+radiation. Mice were treated with siRNA and carboplatin twice a week for 4 weeks and tumor volume was measured biweekly. Tumor weight was measured at the termination of the experiment (FIG. 6A) (Supplementary Table-1). Treatment with control non-targeting siRNA did not inhibit tumor growth as compared to control mice treated with PBS alone (data not shown). The change in tumor volume was significantly different between GFP siRNA and NRF2 siRNA treated tumors (P<0.0001). Tumor weights were significantly higher in the GFP siRNA treated tumors compared to NRF2 siRNA treated tumors (ratio of weights=2.09, 95% CI: [1.41, 3.10], p=0.0002), siRNA compared to siRNA+radiation treated tumors (1.79, 95% CI: [1.18, 2.70], p=0.01), and siRNA compared to siRNA+carboplatin treated tumors (2.13, 95% CI: [1.44, 3.16], p=0.001) (FIG. 6A). The change in tumor volume was significantly different between NRF2 siRNA and GFP siRNA treated tumors (ratio of differences=0.46, 95% CI: [0.31, 0.68], p=0.0001), siRNA+carboplatin and siRNA tumors (0.45, 95% CI: [0.29, 0.71], p=0.0005) and siRNA+radiation and siRNA tumors (0.58, 95% CI: [0.36, 0.95], p=0.03). There was no significant difference in the change in tumor volume between siRNA+carboplatin and siRNA+radiation groups (0.77, 95% CI: [0.45, 1.32], p=0.35). The difference in the change in tumor volume was larger between GFP siRNA+carboplatin and NRF2 siRNA+carboplatin (differences of 352.34 and 58.78) than it was for GFP siRNA and NRF2 siRNA (differences of 532.94 and 249.17), (ratio of differences=2.38, 95% CI: [1.03, 5.48], p=0.042). Data from the second set of experiments validating the same findings is presented in the supplement. (FIG. 14) (Table 3). Gene expression analysis of randomly selected tumors from GFP siRNA and NRF2 siRNA groups demonstrated significant decrease in NRF2 and its downstream target gene expression (FIG. 6B).

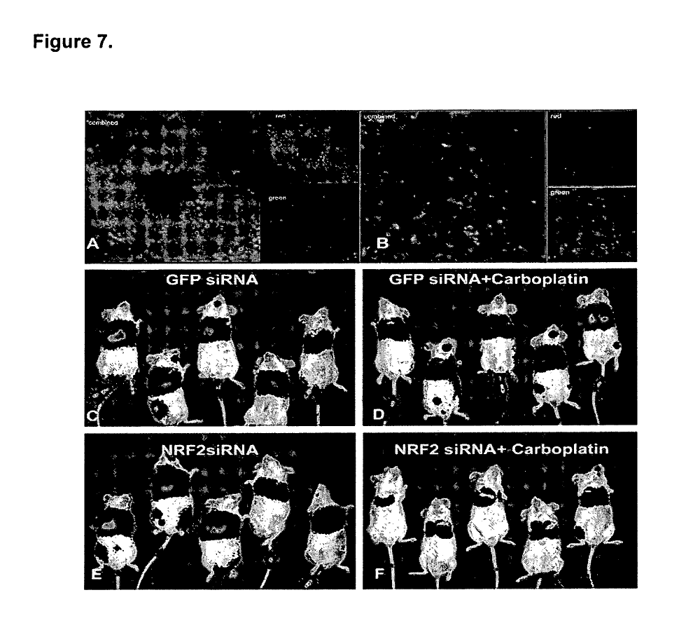

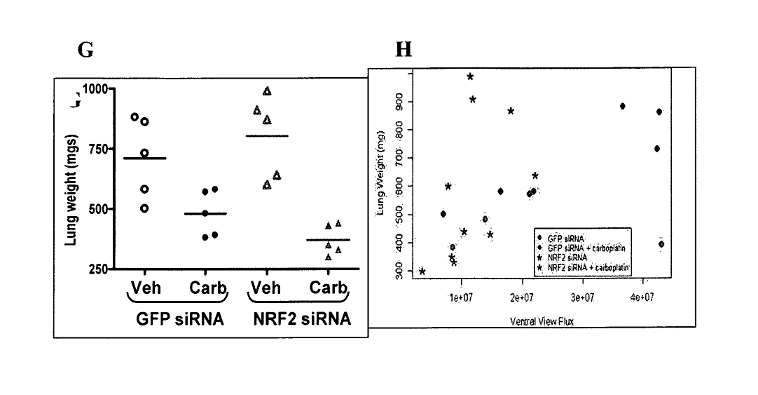

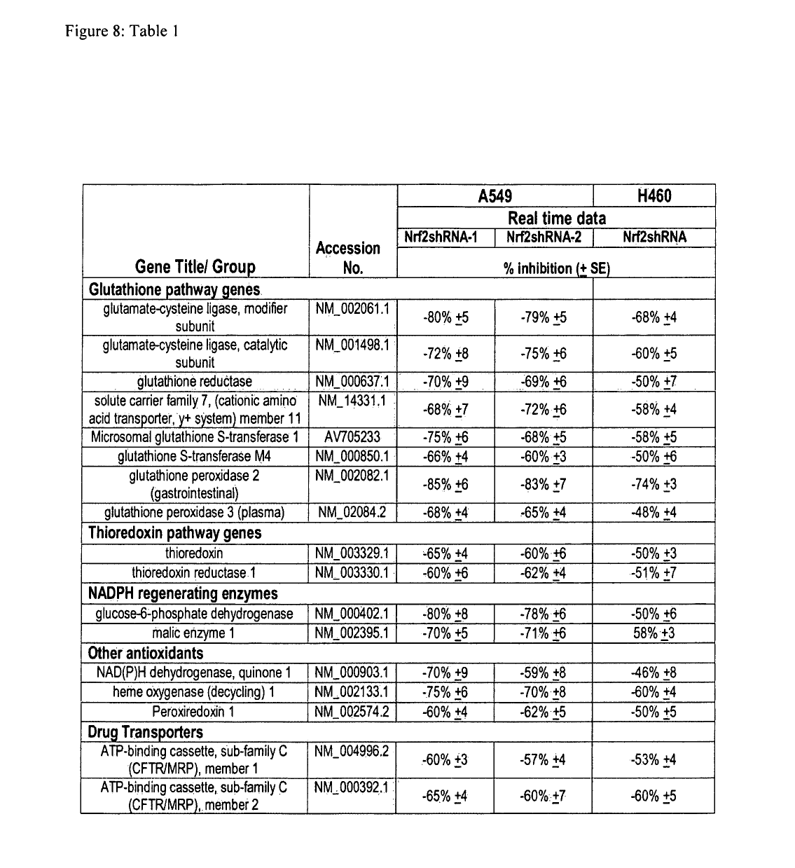

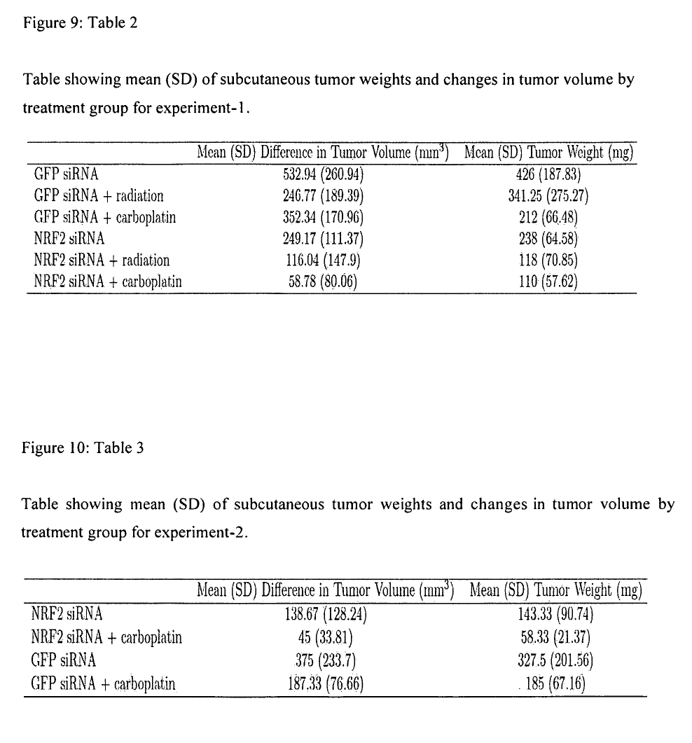

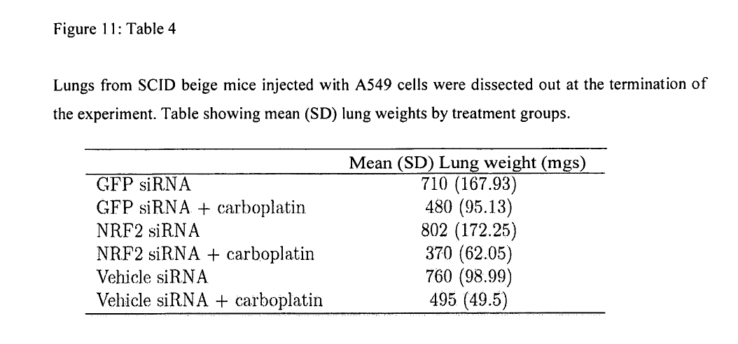

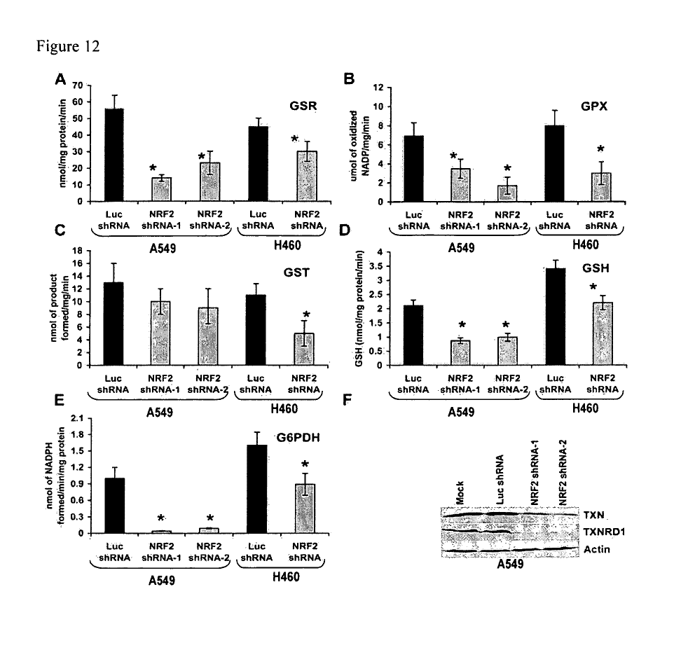

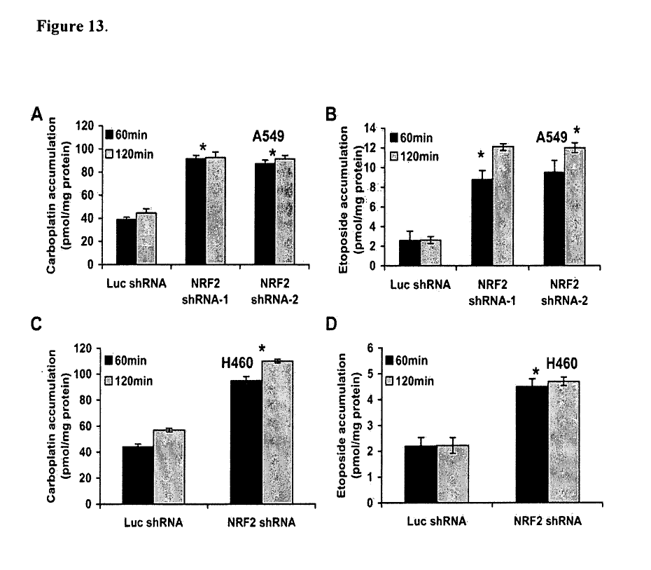

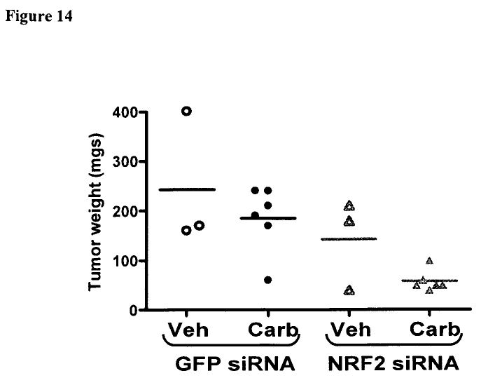

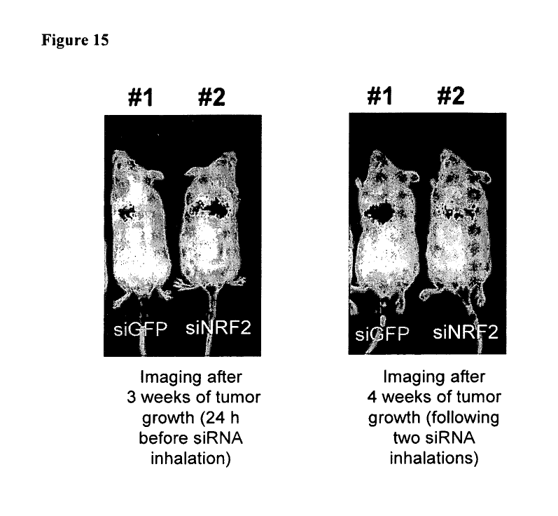

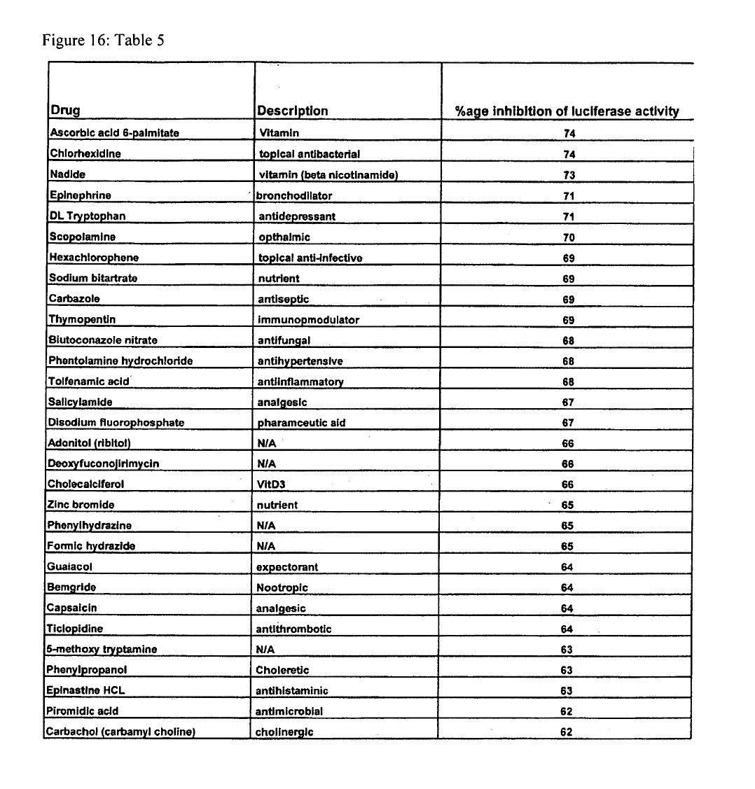

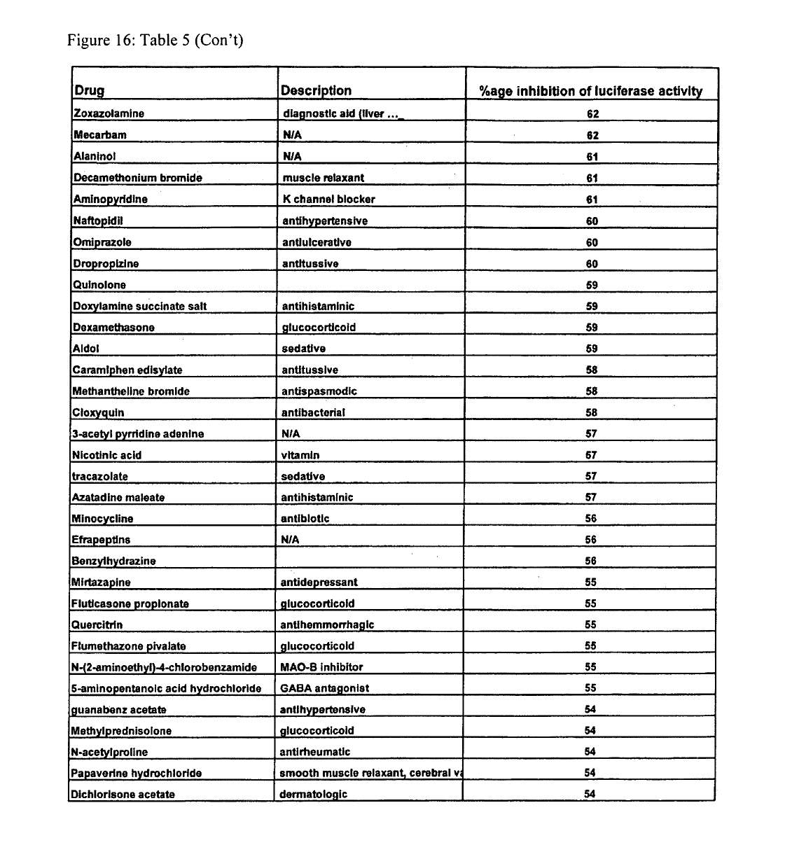

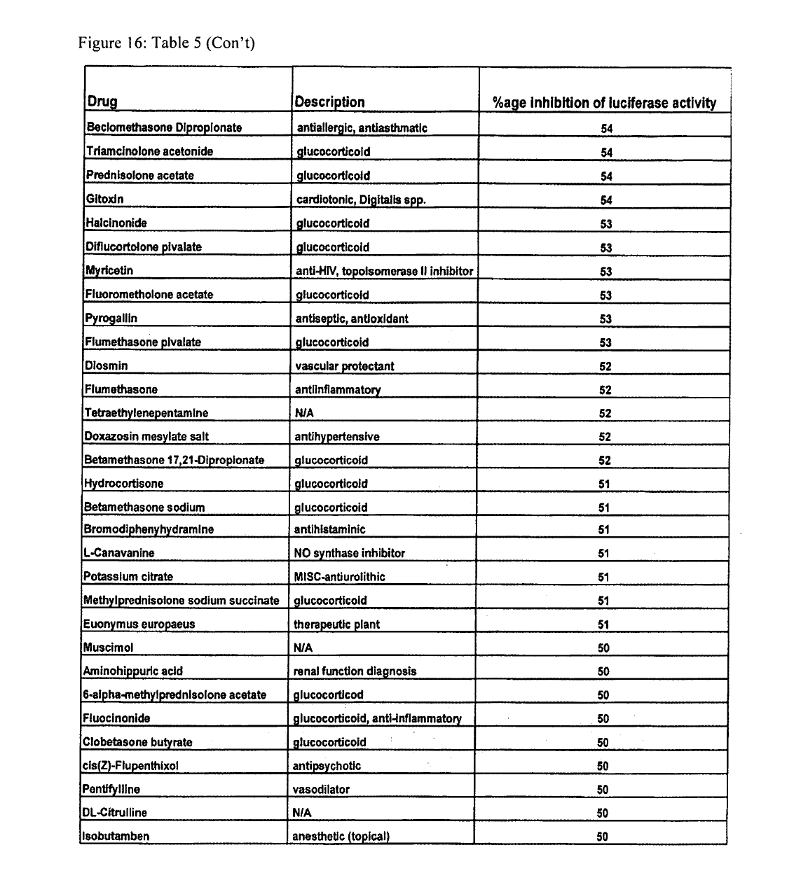

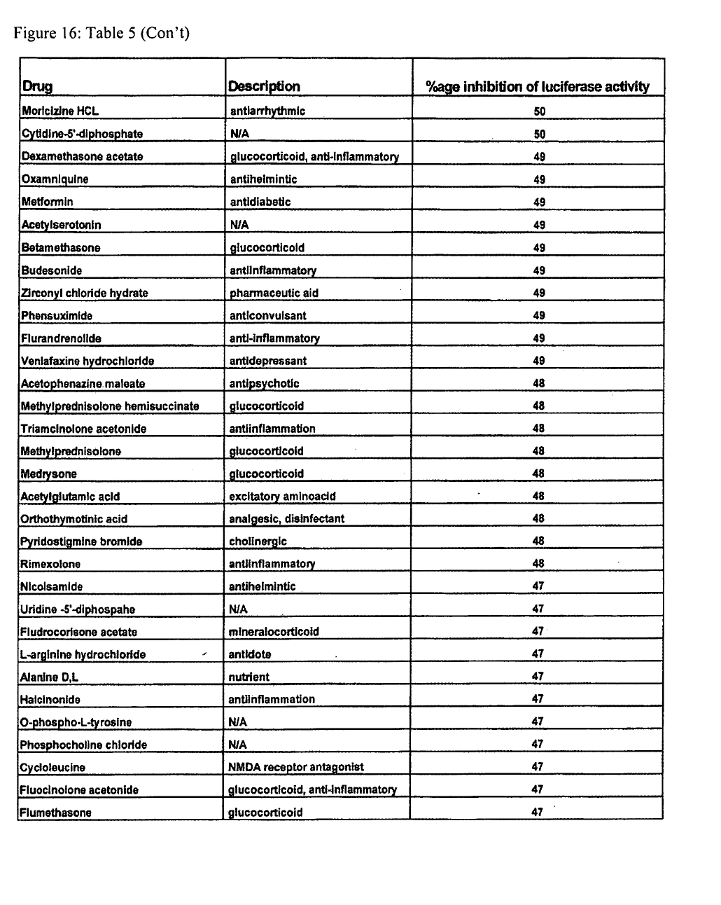

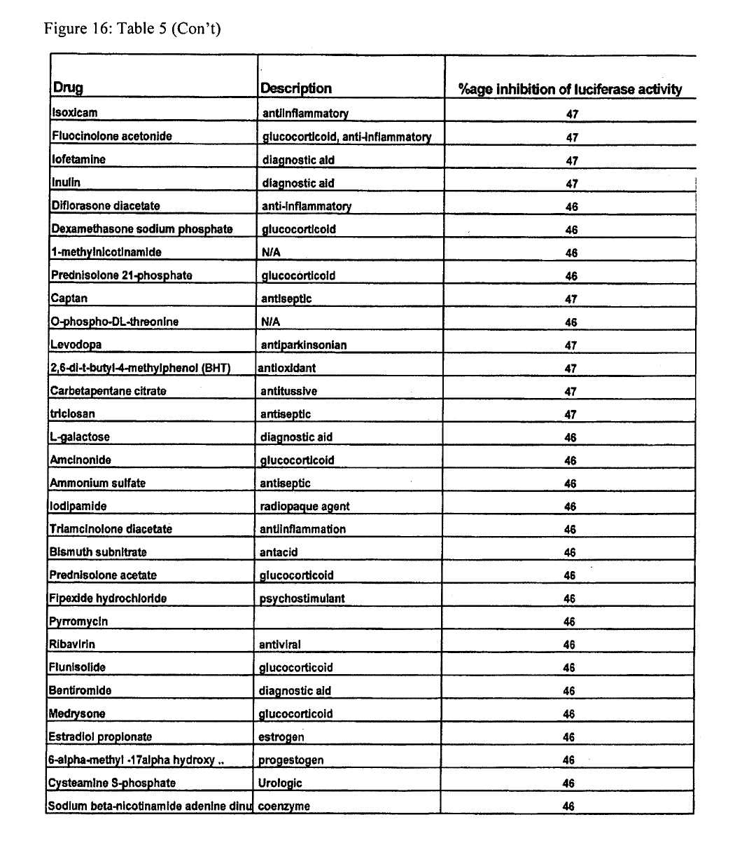

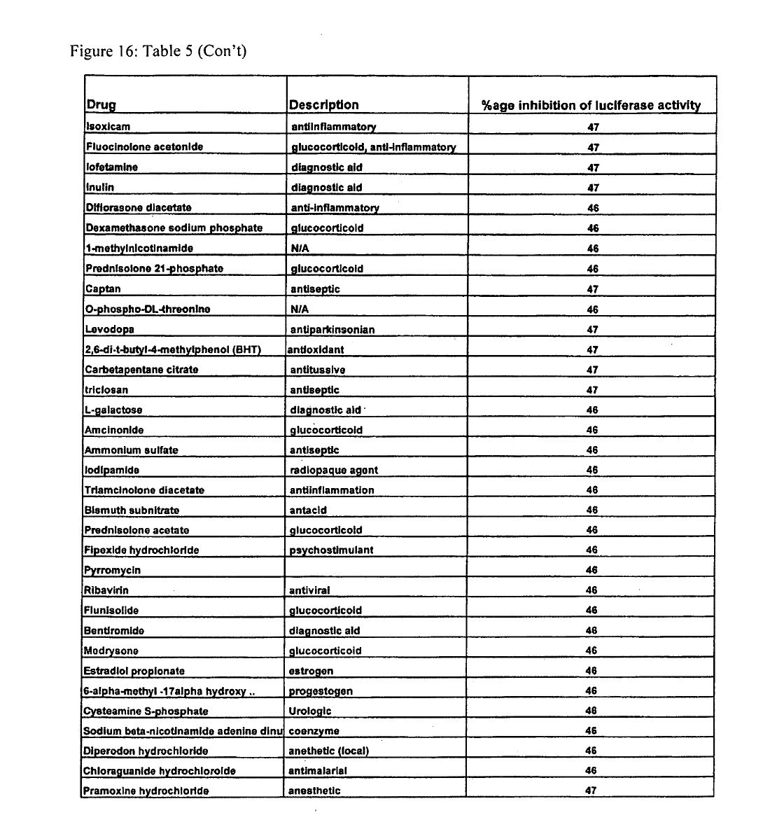

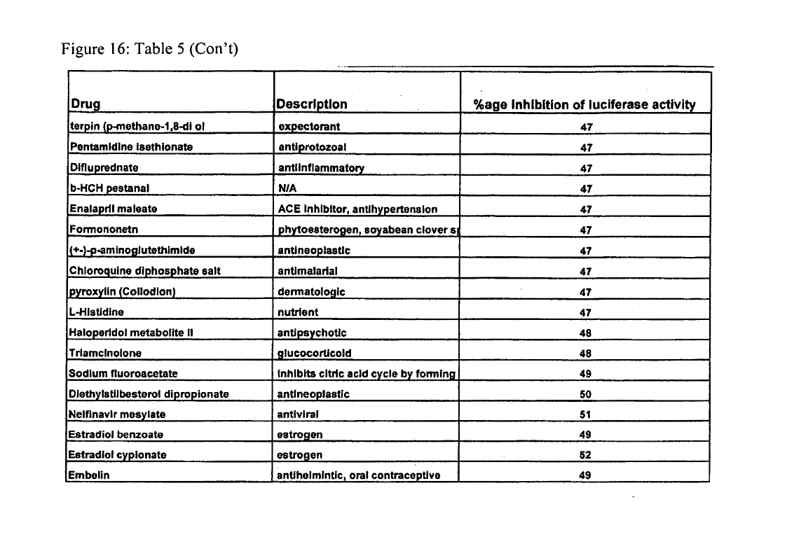

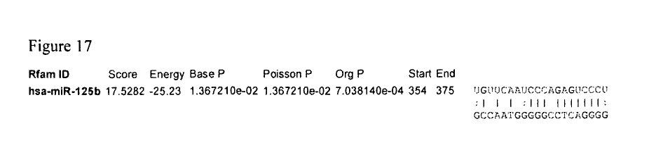

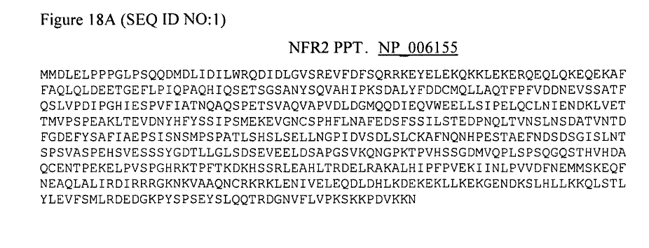

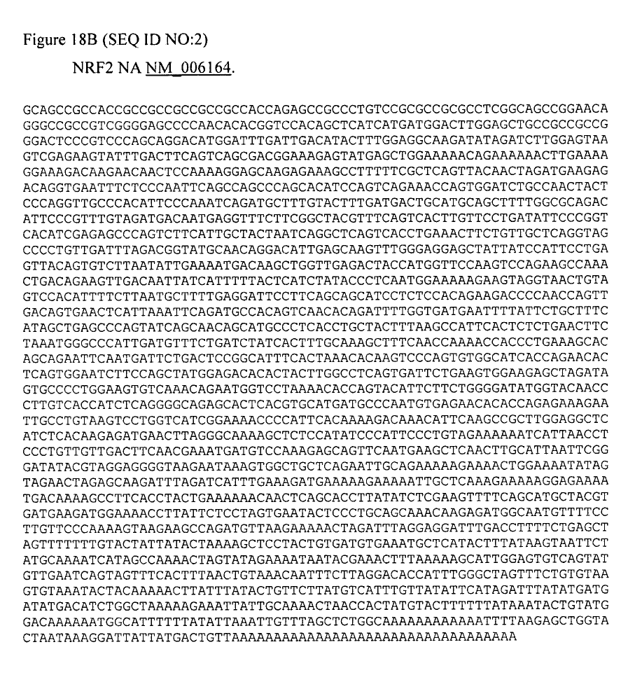

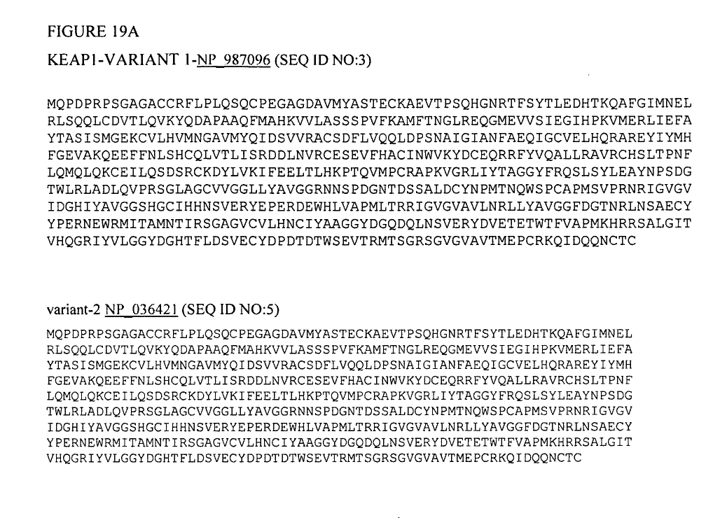

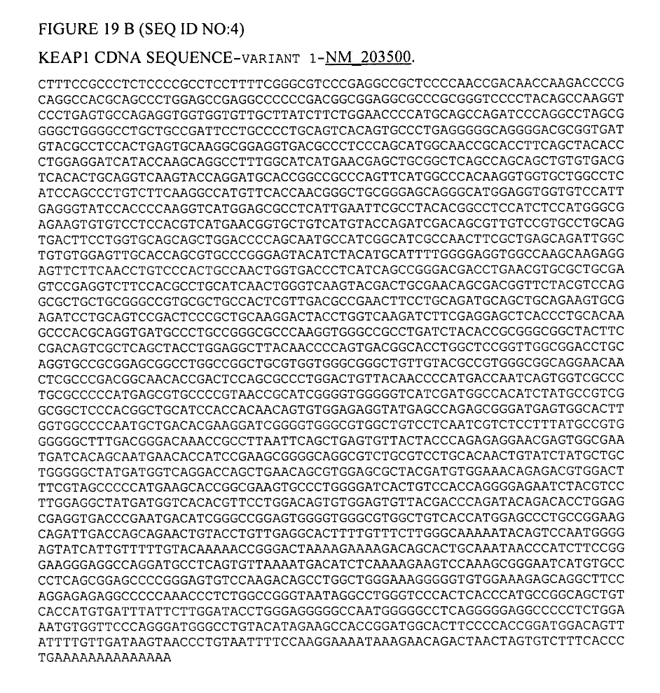

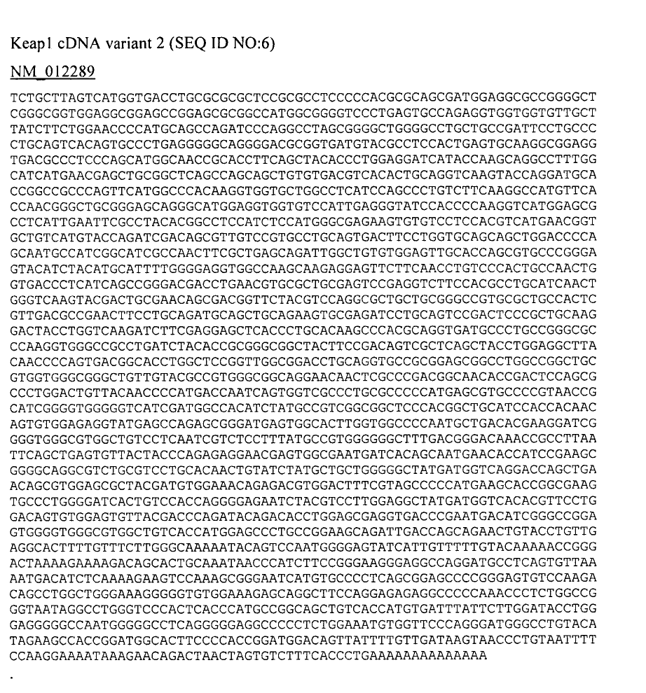

Delivery of naked siRNA duplexes into orthotopic lung tumors: To demonstrate uptake of siRNA by lung tumors, we delivered Cy3 labeled siRNA into mice with lung tumors using a nebulizer. Mice were injected with Lewis lung carcinoma cells and 24 days later (when the mice developed larger tumors) mice were inhaled 100 μg of Cy3 labeled siRNA using a nebulizer. Twenty four hours after siRNA administration, mice were sacrificed; lung harvested and imaged using 2-photon imaging system. There was discrete uptake of Cy3 signal in tissue macrophages throughout the lung parenchyma. Many tumor foci were identified by brightfield and fluorescence microscopy within lung parenchyma (labeled intra-parenchymal tumors). These small intra-parenchymal tumor foci showed robust Cy3 signal. The large surface-protruding tumors showed Cy3 signal but the intensity was several folds lower than that seen in the small intra-parenchymal tumors (FIG. 7A-B).

After successfully delivering labeled siRNA into lung tumors, we tested our hypothesis in an orthotopic model of lung cancer. Mice with lung tumors expressing NRF2-dependent ARE-Luc reporter, were administered two doses of 100 μg of NRF2 siRNA over a period of one week using a nebulizer. Luminescent imaging after 2 doses of NRF2 siRNA delivery demonstrated NRF2 siRNA mediated inhibition of the reporter activity in vivo (FIG. 15). Control mice received non- targeting GFP siRNA.

To study the effect of NRF2 inhibition in combination with carboplatin treatment in an orthotopic model of lung cancer, we injected with A549-C8 luc cells in SCID-Beige mice and randomly allocated to one of the following groups (n=5/group), GFP siRNA, GFP siRNA+carboplatin, NRF2 siRNA and NRF2 siRNA+carboplatin. siRNA inhalations using nebulizer and carboplatin treatment started 1 week after tumor cell injection. After 4 weeks of treatment, mice were imaged using Xenogen imaging system and luciferin substrate (FIG. 7C-F). The lung weights did not vary significantly between overall treatment groups of GFP siRNA and NRF2 siRNA. However, the lung weights for siRNA treated tumors were significantly higher than for siRNA+carboplatin treated tumors (ratio of weights=1.73 [1.46, 2.06], p=0.0001) (FIG. 7G) (Table-4). The difference in weights between siRNA and siRNA+carboplatin treated tumors was significant between NRF2 siRNA and GFP siRNA treated tumors (1.46, 95% CI: [1.03, 2.09], p=0.05). The mean luminescent flux intensities (evaluated by an in vivo imaging) were lowest in mice treated with NRF2 siRNA+carboplatin (FIG. 7H). Thus, combination of NRF2 siRNA with carboplatin/radiation led to a significant reduction in tumor growth after 4 weeks of treatment compared with either agent alone.

### Discussion

Inhibition of NRF2 in A549 and H460 cells resulted in marked decrease in the expression of genes that constitute the glutathione system (GSH biosynthesizing enzymes, glutathione peroxidases (GPx), glutathione reductase (GSR), glutathione S-transferase's (GST's), the thioredoxin system (thioredoxin reductase 1, thioredoxin), peroxiredoxin, NADPH regenerating system (glucose-6-phosphate dehydrogenase, G6PD), antioxidants, and drug efflux pumps (Kensler et al., 2007; Thimmulappa et al., 2002). In corroboration with gene expression, enzyme activities of GSR, GPX, GST and G6PD as well total GSH levels were significantly reduced in A549-NRF2shRNA and H460-NRF2shRNA cells when compared with Luc-shRNA clones. Thus, downregulation of NRF2 expression profoundly decreased the expression of key antioxidant enzymes and electrophile/drug detoxification systems in lung cancer cells with gain of NRF2 function.

Increased reactive oxygen species (ROS) is common in cancer cells and is believed to be attributable at least in part to high metabolism and hyperactive glycolytic metabolism driven by oncogenic proliferative signals (Trachootham et al., 2006). The intrinsic ROS associated with oncogenic transformation renders the cancer cell highly dependent on antioxidant systems to maintain redox balance, and thus, vulnerable to agents that impair antioxidant capacity. The downregulation of NRF2 pathway resulted in dramatic accumulation of intracellular ROS in A549-NRF2shRNA and H460-NRF2shRNA cells. Treatment of these cells with non-specific free radical scavenger N-acetyl cysteine (NAC) for 30 mins reduced ROS production in the A549-NRF2shRNA and H460-NRF2shRNA cells by 85% and 75% respectively. These results suggest that steady state generation of ROS is relatively increased in NRF2 shRNA transfectants as compared to control cancer cells and it provides a biochemical basis to develop new therapeutic strategies to preferentially increase ROS to a toxic level in cancer cells and selectively eradicate them. Interestingly, basal levels of ROS did differ between wild type and Nrf2−/− mouse embryonic fibroblasts (Osburn et al., 2006).

Depletion of NRF2 in both the cancer cell lines resulted in a pronounced decrease in cellular proliferation. Suppression of NRF2 in the H460-NRF2shRNA and A549-NRF2shRNA cells resulted in a substantial reduction in colony formation on soft agar compared to the control cells. Significantly, decreased NRF2 in the A549 cells resulted in complete inhibition of tumor formation in athymic mice whereas H460 cells showed significant reduction in tumor volume and weight. These data indicate that NRF2 is required for growth of cancer cells in vitro and in vivo. Recently, Reddy et at (Reddy et al., 2007) reported that type-II epithelial cells isolated from Nrf2−/− mice lungs display defects in cell proliferation and GSH supplementation rescues these phenotypic defects (Reddy et al., 2007). We hypothesize that decreased antioxidant capacity leading to increased ROS levels in A549-NRF2shRNA and H460-NRF2shRNA cells inhibited the growth of these cells in vitro and in vivo compared to the control A549 and H460 cells expressing Luc-shRNA. Thus, unlike normal cells, constitutive activation of NRF2 is indispensable for maintaining the redox balance and growth of lung cancer cells under homeostatic conditions.

Ionizing radiation triggers the formation of free radicals which interact among themselves and critical biological targets with the formation of a plethora of newer free radicals. It is generally believed that production of these free radicals is the main mechanism through which radiation induces biological damage at lower radiation doses (Weiss and Landauer, 2000). Some of these free radicals damage genomic DNA (Gromer et al., 2004; Kumar et al., 1988; Weiss and Landauer, 2000; Weiss and Landauer, 2003). Antioxidants (glutathione and thioredoxin pathways) and several enzymes such as glutathione-S-transferases, aldehyde dehydrogenases, glutathione peroxidases, thioredoxin and peroxiredoxins constitute the electrophile detoxification system that scavenges the radiation induced electrophiles, thereby causing cellular resistance. Radioprotective effects by modification of antioxidant enzyme expression or by addition of free radical scavengers have been reported (Lee et al., 2004; Tuttle et al., 2000; Weiss and Landauer, 2000; Weiss and Landauer, 2003). Conversely, thiol depletion can result in a higher incidence of radiation induced apoptosis (Mirkovic et al., 1997). In this study, we found that alteration of redox status by NRF2 inhibition enhanced the sensitivity to ionizing radiation through depletion of antioxidants and electrophile detoxification enzymes. Pretreatment with NAC before radiation exposure significantly increased cell survival in A549-NRF2 shRNA and H460-NRF2 shRNA cells. These results clearly indicate that downregulation of NRF2 causes radio sensitization in a ROS-dependent manner.

Anticancer drugs like cisplatin, carboplatin, and oxaliplatin are commonly used intravenous platinating agents. Cisplatin is still used regularly for head and neck and germ cell tumors, while carboplatin has supplanted the use of cisplatin for most ovarian tumors and for the treatment of non-small-cell lung carcinoma (Hartmann and Lipp, 2003; Rabik and Dolan, 2007). Treatment with these agents is characterized by resistance, both acquired and intrinsic. This resistance can be caused by a number of cellular adaptations including reduced uptake, inactivation by glutathione and other antioxidants and increased levels of DNA repair. Since Meister (Meister, 1983) claimed that the cellular metabolism of glutathione could affect the fate of chemotherapeutic agents, several reports have shown that glutathione content is increased in several drug resistant cancer cell lines (Byun et al., 2005; Godwin et al., 1992). Glutathione, a non-protein thiol, can interact via its thiol with the reactive site of a drug, resulting in conjugation of the drug with glutathione. The conjugate is less active and more water soluble and it is excluded from the cell with the participation of transporter proteins named GS-X (including multidrug resistance proteins). Increased levels of glutathione were found in cell lines resistant to alkylating agents (e.g. nitrogen mustard, chlorambucil, melphalan, cyclophosphamide and carmustine) (Tew, 1994). The enzymes glutathione-S-transferases catalyze the interactions between glutathione and alkylating drugs, increasing the rate of a drug detoxification. So, activation of these enzymes can cause cellular drug resistance (Tew, 1994; Zhang et al., 2001). Resistance of tumor cells to drugs vincristine and anthracyclines can also be connected with alterations of the GSH system (Sinha et al., 1989; Tew, 1994). Expression of thioredoxin, another important thiol, increases in many human cancers and is a validated target associated with resistance to standard therapy and decreased patient survival (Powis and Kirkpatrick, 2007). Sasada et al (Sasada et al., 1996) reported that increased expression of thioredoxin contributes to the development of cellular resistance to cisplatin and etoposide (Yokomizo et al., 1995). Inhibition of NRF2 activity by shRNA-mediated gene silencing debilitated the expression of antioxidants and drug detoxification genes thereby increasing the accumulation of etoposide and carboplatin in lung cancer cells and enhanced the cytotoxicity of the drug. Decreased accumulation of these drugs in NRF2 shRNA expressing cells supports the idea that NRF2 contributes to drug resistance by modulating the expression of several drug detoxification enzymes and efflux proteins. Expression of ATP-dependent drug transporters like ABCC1 and ABCC2 were downregulated in the cells expressing NRF2 shRNA.

To elucidate whether the synergistic mode of action of NRF2 shRNA and carboplatin observed in cell culture occurs in vivo, we performed a xenograft experiment with A549 cells. Mice bearing subcutaneous tumors were treated with NRF2 siRNA and carboplatin and tumor volume as well as weight were measured at the termination of the experiment. The tumor weights and volumes were significantly different between GFP siRNA and NRF2 siRNA treated tumors (P=0.0002). Treatment with NRF2 siRNA alone reduced mean tumor weight by 53% (±20% SD) compared to the control group. When NRF2 siRNA was combined with carboplatin, there was an even greater reduction in mean tumor weight in all animals. To explore the effect of radiation exposure in combination with NRF2 inhibition in vivo, we used the same A549 cell xenograft model. In comparison with control siRNA +ionizing radiation treated tumors, combination of NRF2 siRNA plus ionizing radiation produced an additive effect on tumor growth inhibition. We did not observe any synergy between NRF2 siRNA and carboplatin in this in vivo study using limited number of mice. However, similar study with larger sample size needs to be done to determine the potential synergy between NRF2 siRNA and chemotherapeutic drugs in vivo.

After successfully delivering labeled siRNA into lung tumors, we tested our hypothesis in an orthotopic model of lung cancer. Mice with A549 orthotopic lung tumors were treated with siRNA intranasally using a nebulizer followed by carboplatin treatment. Mice receiving NRF2 siRNA along with carboplatin demonstrated significantly higher growth inhibition when compared with control mice receiving GFP siRNA along with carboplatin. Thus, combination of NRF2 siRNA with carboplatin/radiation led to a significant reduction in tumor growth compared with either agent alone. This study suggests that NRF2 siRNA inhibitors are highly efficient promoters for the antineoplastic potential of drugs such as carboplatin, causing additive/synergistic effects in cancer cells.

Several miRNA's targeting KEAP1 have been identified. These are hsa-miR-125b, hsa-miR-491 and has-miR-141. These miRNA's inhibit KEAP1 activity leading to activation of NRF2 pathway. Antimers or small molecules targeting KEAP1 miRNA also can be used to inhibit NRF2 activity.

### EXAMPLE 2

### A Novel Assay for Nrf2 Inhibitors

A high throughput approach to screen compounds was developed. A cell based reporter assay was used to identify agents that can inhibit Nrf2 mediated transcription. Lung adenocarcinoma cells that are stably transfected with ARE-luciferase reporter vector were plated on to 96 well plates. After overnight incubation, cells were pretreated with 12-16 hours with candidate Nrf2 modulators. Luciferase activity was measured after 6 hours of treatment using a luciferase assay system. The decrease in luciferase activity correlates with degree of Nrf2 inhibition.

The compounds identified using this assay are identified in FIG. 16: Table 5. The known use of each compound is identified in the middle column and the percent luciferase activity is identified in the right hand column.

### REFERENCES

Byun, S. S., Kim, S. W., Choi, H., Lee, C., and Lee, E. (2005). Augmentation of cisplatin sensitivity in cisplatin-resistant human bladder cancer cells by modulating glutathione concentrations and glutathione-related enzyme activities. BJU Int 95, 1086-1090.

Godwin, A. K., Meister, A., O'Dwyer, P. J., Huang, C. S., Hamilton, T. C., and Anderson, M. E. (1992). High resistance to cisplatin in human ovarian cancer cell lines is associated with marked increase of glutathione synthesis. Proc Natl Acad Sci U S A 89, 3070-3074.

Gromer, S., Urig, S., and Becker, K. (2004). The thioredoxin system--from science to clinic. Med Res Rev 24, 40-89.

Hamar, P., Song, E., Kokeny, G., Chen, A., Ouyang, N., and Lieberman, J. (2004). Small interfering RNA targeting Fas protects mice against renal ischemia-reperfusion injury. Proc Natl Acad Sci U S A 101, 14883-14888.

Hartmann, J. T., and Lipp, H. P. (2003). Toxicity of platinum compounds. Expert Opin Pharmacother 4, 889-901.

Hayashi, A., Suzuki, H., Itoh, K., Yamamoto, M., and Sugiyama, Y. (2003). Transcription factor Nrf2 is required for the constitutive and inducible expression of multidrug resistance-associated protein 1 in mouse embryo fibroblasts. Biochem Biophys Res Commun 310, 824-829.

Kensler, T. W., Wakabayashi, N., and Biswal, S. (2007). Cell survival responses to environmental stresses via the Keap1-Nrf2-ARE pathway Annu Rev Pharmacol Toxicol 47, 89-116.

Kim, Y. J., Ahn, J. Y., Liang, P., Ip, C., Zhang, Y., and Park, Y. M. (2007). Human prx1 gene is a target of Nrf2 and is up-regulated by hypoxia/reoxygenation: implication to tumor biology. Cancer Res 67, 546-554.

Kobayashi, A., Kang, M. I., Okawa, H., Ohtsuji, M., Zenke, Y., Chiba, T., Igarashi, K., and Yamamoto, M. (2004). Oxidative stress sensor Keap1 functions as an adaptor for Cu13-based E3 ligase to regulate proteasomal degradation of Nrf2. Mol Cell Biol 24, 7130-7139.

Kotlo, K. U., Yehiely, F., Efimova, E., Harasty, H., Hesabi, B., Shchors, K., Einat, P., Rozen, A., Berent, E., and Deiss, L. P. (2003). Nrf2 is an inhibitor of the Fas pathway as identified by Achilles' Heel Method, a new function-based approach to gene identification in human cells. Oncogene 22, 797-806.

Kumar, K. S., Vaishnav, Y. N., and Weiss, J. F. (1988). Radioprotection by antioxidant enzymes and enzyme mimetics. Pharmacol Ther 39, 301-309.

Kurosu, T., Fukuda, T., Miki, T., and Miura, O. (2003). BCL6 overexpression prevents increase in reactive oxygen species and inhibits apoptosis induced by chemotherapeutic reagents in B-cell lymphoma cells. Oncogene 22, 4459-4468.

Lee, H. C., Kim, D. W., Jung, K. Y., Park, I. C., Park, M. J., Kim, M. S., Woo, S. H., Rhee, C. H., Yoo, H., Lee, S. H., and Hong, S. I. (2004). Increased expression of antioxidant enzymes in radioresistant variant from U251 human glioblastoma cell line. Int J Mol Med 13, 883-887.

Lee, T. D., Yang, H., Whang, J., and Lu, S. C. (2005). Cloning and characterization of the human glutathione synthetase 5′-flanking region. Biochem J 390, 521-528.

Masuda, H., Tanaka, T., and Takahama, U. (1994). Cisplatin generates superoxide anion by interaction with DNA in a cell-free system. Biochem Biophys Res Commun 203, 1175-1180.

Meister, A. (1983). Selective modification of glutathione metabolism. Science 220, 472-477.

Mirkovic, N., Voehringer, D. W., Story, M. D., McConkey, D. J., McDonnell, T. J., and Meyn, R. E. (1997). Resistance to radiation-induced apoptosis in Bc1-2-expressing cells is reversed by depleting cellular thiols. Oncogene 15, 1461-1470.

Morito, N., Yoh, K., Itoh, K., Hirayama, A., Koyama, A., Yamamoto, M., and Takahashi, S. (2003). Nrf2 regulates the sensitivity of death receptor signals by affecting intracellular glutathione levels. Oncogene 22, 9275-9281.

Nadkar, A., Pungaliya, C., Drake, K., Zajac, E., Singhal, S. S., and Awasthi, S. (2006). Therapeutic resistance in lung cancer. Expert Opin Drug Metab Toxicol 2, 753-777.

Nguyen, T., Sherratt, P. J., and Pickett, C. B. (2003). Regulatory mechanisms controlling gene expression mediated by the antioxidant response element Annu Rev Pharmacol Toxicol 43, 233-260.

Osburn, W. O., Wakabayashi, N., Misra, V., Nilles, T., Biswal, S., Trush, M. A., and Kensler, T. W. (2006). Nrf2 regulates an adaptive response protecting against oxidative damage following diquat-mediated formation of superoxide anion. Arch Biochem Biophys 454, 7-15.

Padmanabhan, B., Tong, K. I., Ohta, T., Nakamura, Y., Scharlock, M., Ohtsuji, M., Kang, M. I., Kobayashi, A., Yokoyama, S., and Yamamoto, M. (2006). Structural basis for defects of Keap1 activity provoked by its point mutations in lung cancer. Mol Cell 21, 689-700.

Powis, G., and Kirkpatrick, D. L. (2007). Thioredoxin signaling as a target for cancer therapy. Curr Opin Pharmacol.

Rabik, C. A., and Dolan, M. E. (2007). Molecular mechanisms of resistance and toxicity associated with platinating agents. Cancer Treat Rev 33, 9-23.

Rangasamy, T., Cho, C. Y., Thimmulappa, R. K., Zhen, L., Srisuma, S. S., Kensler, T. W., Yamamoto, M., Petrache, I., Tuder, R. M., and Biswal, S. (2004). Genetic ablation of Nrf2 enhances susceptibility to cigarette smoke-induced emphysema in mice. J Clin Invest 114, 1248-1259.

Rangasamy, T., Guo, J., Mitzner, W. A., Roman, J., Singh, A., Fryer, A. D., Yamamoto, M., Kensler, T. W., Tuder, R. M., Georas, S. N., and Biswal, S. (2005). Disruption of Nrf2 enhances susceptibility to severe airway inflammation and asthma in mice. J Exp Med 202, 47-59.

Reddy, N. M., Kleeberger, S. R., Cho, H. Y., Yamamoto, M., Kensler, T. W., Biswal, S., and Reddy, S. P. (2007). Deficiency in Nrf2-GSH signaling impairs type II cell growth and enhances sensitivity to oxidants. Am J Respir Cell Mol Biol 37, 3-8.

Sasada, T., Iwata, S., Sato, N., Kitaoka, Y., Hirota, K., Nakamura, K., Nishiyama, A., Taniguchi, Y., Takabayashi, A., and Yodoi, J. (1996). Redox control of resistance to cis-diamminedichloroplatinum (II) (CDDP): protective effect of human thioredoxin against CDDP-induced cytotoxicity. J Clin Invest 97, 2268-2276.

Singh, A., Misra, V., Thimmulappa, R. K., Lee, H., Ames, S., Hogue, M. O., Herman, J. G., Baylin, S. B., Sidransky, D., Gabrielson, E., et al. (2006a). Dysfunctional KEAP1-NRF2 interaction in non-small-cell lung cancer. PLoS Med 3, e420.

Singh, A., Rangasamy, T., Thimmulappa, R. K., Lee, H., Osburn, W. O., Brigelius-Flohe, R., Kensler, T. W., Yamamoto, M., and Biswal, S. (2006b). Glutathione peroxidase 2, the major cigarette smoke-inducible isoform of GPX in lungs, is regulated by Nrf2. Am J Respir Cell Mol Biol 35, 639-650.

Sinha, B. K., Mimnaugh, E. G., Rajagopalan, S., and Myers, C. E. (1989). Adriamycin activation and oxygen free radical formation in human breast tumor cells: protective role of glutathione peroxidase in adriamycin resistance. Cancer Res 49, 3844-3848.

Sjoblom, T., Jones, S., Wood, L. D., Parsons, D. W., Lin, J., Barber, T. D., Mandelker, D., Leary, R. J., Ptak, J., Silliman, N., et al. (2006). The consensus coding sequences of human breast and colorectal cancers. Science 314, 268-274.

Soini, Y., Napankangas, U., Jarvinen, K., Kaarteenaho-Wiik, R., Paakko, P., and Kinnula, V. L. (2001). Expression of gamma-glutamyl cysteine synthetase in nonsmall cell lung carcinoma. Cancer 92, 2911-2919.

Tew, K. D. (1994). Glutathione-associated enzymes in anticancer drug resistance. Cancer Res 54, 4313-4320.

Thimmulappa, R. K., Mai, K. H., Srisuma, S., Kensler, T. W., Yamamoto, M., and Biswal, S. (2002). Identification of Nrf2-regulated genes induced by the chemopreventive agent sulforaphane by oligonucleotide microarray. Cancer Res 62, 5196-5203.

Thimmulappa, R. K., Scollick, C., Traore, K., Yates, M., Trush, M. A., Liby, K. T., Sporn, M. B., Yamamoto, M., Kensler, T. W., and Biswal, S. (2006). Nrf2-dependent protection from LPS induced inflammatory response and mortality by CDDO-Imidazolide. Biochem Biophys Res Commun 351, 883-889.

Trachootham, D., Zhou, Y., Zhang, H., Demizu, Y., Chen, Z., Pelicano, H., Chiao, P. J., Achanta, G., Arlinghaus, R. B., Liu, J., and Huang, P. (2006). Selective killing of oncogenically transformed cells through a ROS-mediated mechanism by beta-phenylethyl isothiocyanate. Cancer Cell 10, 241-252.

Tsutomu Ohta, K. I., Mamiko Miyamoto, Izumi Nakahara, Hiroshi Tanaka, Makiko Ohtsuji, Takafumi Suzuki, Akira Kobayashi, Jun Yokota, Tokuki Sakiyama, Tatsuhiro Shibata, Masayuki Yamamoto, and Setsuo Hirohashi (2008). Loss of Keap1 Function Activates Nrf2 and Provides Advantages for Lung Cancer Cell Growth Cancer Res 68, 1303-1309.

Tuttle, S., Stamato, T., Perez, M. L., and Biaglow, J. (2000). Glucose-6-phosphate dehydrogenase and the oxidative pentose phosphate cycle protect cells against apoptosis induced by low doses of ionizing radiation. Radiat Res 153, 781-787.

Vollrath, V., Wielandt, A. M., Iruretagoyena, M., and Chianale, J. (2006). Role of Nrf2 in the regulation of the Mrp2 (ABCC2) gene. Biochem J 395, 599-609.

Weiss, J. F., and Landauer, M. R. (2000). Radioprotection by antioxidants. Ann N Y Acad Sci 899, 44-60.

Weiss, J. F., and Landauer, M. R. (2003). Protection against ionizing radiation by antioxidant nutrients and phytochemicals. Toxicology 189, 1-20.

Yang, P., Ebbert, J. O., Sun, Z., and Weinshilboum, R. M. (2006). Role of the glutathione metabolic pathway in lung cancer treatment and prognosis: a review. J Clin Oncol 24, 1761-1769.

Yokomizo, A., Ono, M., Nanri, H., Makino, Y., Ohga, T., Wada, M., Okamoto, T., Yodoi, J., Kuwano, M., and Kohno, K. (1995). Cellular levels of thioredoxin associated with drug sensitivity to cisplatin, mitomycin C, doxorubicin, and etoposide. Cancer Res 55, 4293-4296.

Zhang, D. D., Lo, S. C., Cross, J. V., Templeton, D. J., and Hannink, M. (2004). Keap1 is a redox-regulated substrate adaptor protein for a Cu13-dependent ubiquitin ligase complex. Mol Cell Biol 24, 10941-10953.

Zhang, K., Chew, M., Yang, E. B., Wong, K. P., and Mack, P. (2001). Modulation of cisplatin cytotoxicity and cisplatin-induced DNA cross-links in HepG2 cells by regulation of glutathione-related mechanisms. Mol Pharmacol 59, 837-843.

1. A Nrf2 inhibitor as set forth in Table 5.

2-7. (canceled)

8. A method for identifying an inhibitor of Nrf2 comprising:
contacting a carcinoma cell transfected with luciferase with a candidate inhibitor of Nrf2; and
measuring the luciferase activity in the cells;
wherein a decrease in the amount of luciferase activity as compared to a carcinoma cell not contacted with the candidate inhibitor is indicative of the candidate inhibitor being an inhibitor of Nrf2.

9. The method of claim 8, wherein the carcinoma cell is a adenocarcinoma cell.

10-12. (canceled)

13. A method of treating a subject having a cell proliferative disorder, comprising:
administering to the subject an effective amount of a Nrf2 inhibitor;
thereby treating the subject.

14. The method of claim 13, wherein the subject is administered an additional anticancer treatment.

15. The method of claim 14, wherein the anticancer treatment is radiation or a chemotherapeutic.

16. The method of claim 13, wherein the cell proliferative disorder is cancer.

17-20. (canceled)

21. A method of treating a subject having a cell proliferative disorder comprising:
administering to the subject a Nrf2 inhibitor and one or more additional anticancer treatments,
thereby treating the subject.

22. The method of claim 21, wherein the anticancer treatment is radiation or a chemotherapeutic.

23. The method of claim 22, wherein the cell proliferative disorder is cancer.

24-28. (canceled)

29. A method of treating a subject having a cell proliferative disorder comprising:
administering to the subject a compound that inhibits the expression or activity of Nrf2;
thereby treating the subject.

30-37. (canceled)

38. A method of determining if a subject is at risk of becoming resistant to an anticancer treatment comprising:
determining if a subject has a mutation in the KEAP1 gene;
thereby determining if a subject is at risk of developing resistance to anticancer treatment.

39. The method of claim 38, wherein the anticancer treatment is a chemotherapeutic or radiation.

40. The method of claim 38, wherein the mutation results in an amino acid substitution.

41. The method of claim 40, wherein the mutation results in an amino acid substitution at position 255 KEAP1.

42. The method of claim 41, wherein the mutation is a Tyr to His mutation.

43. The method of claim 42, wherein the mutation results in an amino acid substitution at position 314 KEAP1.

44. The method of claim 43, wherein the mutation is a Thr to Met mutation.

45. (canceled)

46. A pharmaceutical composition for the treatment of cancer comprising a Nrf2 inhibitor and a pharmaceutically acceptable carrier, or
a pharmaceutical composition comprising one or more Nrf2 inhibitors, one or more additional anticancer compositions and a pharmaceutically acceptable carrier, or
a kit for identifying inhibitors of Nrf2 comprising a carcinoma cell transfected with luciferase and instructions for use.

47-51. (canceled)
